(19) United States
(12) Patent Application Publication
(10) Pub. No.: US 2003/0119816 A1
(43) Pub. Date: Jun. 26, 2003

(54) Flavone derivatives, preparation method and use as medicines

(76) Inventors: Jean-Luc Haesslein, Courtry (FR); Dominique Lefrancois, Le Raincy (FR); Eric Uridat, Pommiers (FR); Jidong Zhang, Paris (FR)

Correspondence Address: Bierman Muserlian & Lucas, 600 Third Avenue, New York, NY, 10016, US

(21) Appl. No.: 10/181,677

(22) PCT Filed: Feb. 27, 2001
(86) PCT No.: PCT/FR01/00561

(30) Foreign Application Priority Data
Feb. 29, 2000 (FR) 00 02528

(51) Int. Cl. C07D 417/02; C07D 413/02; C 07D 4 5/02; A61K 31/55; A61K 31/541; A61K 31/5377; A61K 31/496; A61K 31/453; A61K 31/4025; A61K 31/353

(52) U.S. Cl. 514/217.03; 514/228.2; 514/254.11; 514/232.8; 514/422; 514/320; 514/456; 544/60; 540/596; 544/151; 544/376; 546/196; 548/525; 549/403

(57) ABSTRACT

The invention concerns novel products of formula (I) wherein: R1 represents a carbocyclic or heterocyclic radical optionally substituted; R2 and R3 are such that one represents hydrogen and the other represents piperidinyl optionally substituted; R4 represents hydrogen, alkyl or phenyl optionally substituted, said products being in all isomeric and salt forms and used as medicines.

The present invention relates to new derivatives of flavones, their preparation process, new deprotection method for the methyl ethers, the new intermediates obtained, their use as medicaments, the pharmaceutical compositions containing them and the new use of such derivatives of flavones.

Therefore a subject of the invention is new derivatives of flavones having anti-proliferative properties and in particular derivatives of flavones endowed with an inhibitory effect vis-a-vis the cycline-dependent kinase proteins i.e. abbreviated to ‘cdk’ which will be used in the rest of the text.

Study of the molecular mechanisms which control the cell cycle has allowed the regulatory role of the cdk's thus defined to be demonstrated. The cdk's are proteins constituted by at least two sub-units, a catalytic sub-unit (of which cdc2 is the prototype) and a regulatory sub-unit (cycline). Thus a certain number of cdk's are known. The cdk's therefore form proteinic complexes, each of which is involved in a phase of the cell cycle.

Numerous documents in the literature describe the existence and the role of cdk's and by way of example, in particular the document WO 97/20842 can be mentioned.

Several inhibitors of kinases have been described such as butyrolactone, flavopiridol and 2(2-hydroxyethylamino)-6-benzylamino-9-methylpurine called olomoucine.

Therefore a subject of the present invention is the products of formula (I):

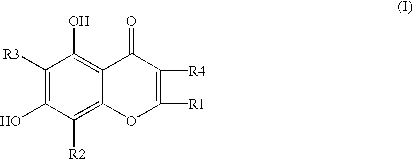

in which:

R1 represents a carbocyclic or heterocyclic monocyclic or bicyclic radical containing at most 12 members, saturated or unsaturated, containing one or more identical or different heteroatoms chosen from 0, N, NH or S and can contain a —C(O) member,

the carbocyclic and heterocyclic radicals as defined above for R1, being optionally substituted by one or more radicals chosen from halogen atoms; the hydroxyl; cycloalkyl containing at most 6 members; acyl containing at most 7 carbon atoms; cyano; nitro; free, salified or esterified carboxy; tetrazolyl; —NH2, —NH(alk), —N(alk)(alk); SO2-NH—CO—NHR5 in which R5 represents an alkyl or phenyl radical; —C(O)—NH2 , —C(O)—NH(alk), —C(O)—N(alk)(alk), —NH—C(O)-(alk), —N(alk)-C(O)-(alk); thienyl; phenyl; alkylphenyl; alkyl, alkenyl, alkylthio, alkoxy or phenoxy radicals themselves optionally substituted by one or more radicals chosen from halogen atoms, the —NH2, —NH(alk), —N(alk)(alk) radicals and the heterocyclic monocyclic or bicyclic radicals themselves optionally substituted by one or more radicals chosen from alkyl radicals and halogen atoms,

R2 and R3 are such that one represents a hydrogen atom and the other represents a piperidinyl radical optionally substituted by one or more hydroxyl and alkyl radicals, R2 and R3 being alternatively able to take the same values in order to produce the corresponding isomers,

R4 represents a hydrogen atom, an alkyl or phenyl radical optionally substituted by one or more halogen atoms. it being understood that in the above radicals, the alkyl and alkoxy radicals are linear or branched and contain at most 6 carbon atoms,

said products of formula (I) being in all possible racemic, enantiomeric and diastereoisomeric isomer forms, as well as the addition salts with mineral and organic acids or with mineral and organic bases of said products of formula (I).

In the products of formula (I) and in what follows:

the term linear or branched alkyl radical designates the methyl, ethyl, propyl, isopropyl, butyl, isobutyl, sec-butyl, tert-butyl, pentyl, isopentyl, hexyl, isohexyl and also heptyl, octyl, nonyl and decyl radicals as well as their linear or branched position isomers,

the term linear or branched alkoxy designates the methoxy, ethoxy, propoxy, isopropoxy, linear, secondary or tertiary butoxy, pentoxy or hexoxy radicals as well as their linear or branched position isomers,

the term halogen designates the chlorine, bromine, iodine or fluorine atoms and preferably the chlorine, bromine or fluorine atom,

The term carbocyclic or heterocyclic monocyclic or bicyclic radical containing at most 12 members, saturated or unsaturated, containing one or more identical or different heteroatoms chosen from 0, N, NH or S, and can contain a-C(O) member, brings together the following definitions:

the term unsaturated carbocyclic radical designates in particular a cycloalkyl radical

the term cycloalkyl radical designates the cyclopropyl, cyclobutyl, cyclopentyl and cyclohexyl radicals and quite particularly the cyclopentyl and cyclohexyl radicals,

the term heterocyclic monocyclic radical designates a saturated or unsaturated radical constituted by 5 or 6 members such that one or more of the members represents an oxygen, sulphur or nitrogen atom: such a heterocyclic radical thus designates a carbocyclic radical interrupted by one or more heteroatoms chosen from oxygen, nitrogen or sulphur atoms it being understood that the heterocyclic radicals can contain one or more heteroatoms chosen from oxygen, nitrogen or sulphur atoms and that when these heterocyclic radicals comprise more than one heteroatom, the heteroatoms of these heterocyclic radicals can be identical or different. There can be mentioned in particular the following radicals: dioxolane, dioxane, dithiolane, thiooxolane, thiooxane, morpholinyl, piperazinyl, piperazinyl substituted by a linear or branched alkyl radical containing at most 4 carbon atoms, piperidyl, thienyl such as 2-thienyl and 3-thienyl, furyl such as 2-furyl, pyrimidinyl, pyridyl such as 2-pyridyl, 3-pyridyl and 4-pyridyl pyrimidyl, pyrazolinyl, pyrrolyl, thiazolyl, isothiazolyl, diazolyl, thiadiazolyl, triazolyl, tetrazolyl, free or salified thiadiazolyl, thiatriazolyl, oxazolyl, oxadiazolyl, 3- or 4-isoxazolyl. There can be quite particularly mentioned the morpholinyl, thienyl such as 2-thienyl and 3-thienyl, furyl such as 2-furyl, tetrahydrofuryl, thienyl, tetrahydrothienyl, pyrrolyl, pyrrolinyl, pyrazolinyl, isoxazolyl, pyridyl and pyrrolidinyl radicals.

the term heterocyclic bicyclic radical designates a saturated or unsaturated radical constituted by 8 to 12 members such that one or more of the members represents an oxygen, sulphur or nitrogen atom and in particular the condensed heterocyclic groups containing at least one heteroatom chosen from sulphur, nitrogen and oxygen, for example benzothienyl such as 3-benzothienyl, benzothiazolyl, quinolyl, isoquinolyl, tetralone, benzofuryl, benzopyrrolyl, benzimidazolyl, benzoxazolyl, thionaphthyl, indolyl or purinyl.

the term saturated carbocyclic radical designates the phenyl and naphthyl radicals and in particular the phenyl radical. It can be noted that a carbocyclic radical containing a —C(O) member is for example the tetralone radical.

the term alkylphenyl designates a phenyl radical substituted by one or more linear or branched alkyl radicals as defined above preferably containing at most 4 carbon atoms

the terms NH(alk) and N(alk)(alk) designate an amino radical substituted respectively by one or two alkyl radicals, such alkyl radicals being linear or branched and preferably containing at most 4 carbon atoms

the term acylamino designates the —C(O)—NH2, —C(O)—NH(alk) and —C(O)—N(alk)(alk) radicals: in these radicals, NH(alk) and N(alk)(alk) have the meanings indicated above

the term acyl designates an R—C(O)— radical in which R represents a radical chosen from the hydrogen atom, the linear or branched alkyl radicals containing at most 6 carbon atoms, a phenyl radical or a pyrrolidinyl radical: the term acyl thus designates in particular the formyl radicals, the acetyl, propionyl, butanoyl, pentanoyl, hexanoyl, benzoyl and pyrrolidinylcarbonyl radicals

the term alkenyl designates linear or branched radicals containing at most 6 carbon atoms: there can be mentioned in particular the vinyl, 1-propenyl, allyl, butenyl, 3-methyl-2-butenyl radicals

the term alkylthio designates linear or branched radicals containing at most 6 carbon atoms such as in particular the methylthio, ethylthio, propylthio, isopropylthio, butylthio, isobutylthio, sec-butylthio, tert-butylthio, pentylthio, isopentylthio, hexylthio or also isohexylthio radicals as well as their linear or branched position isomers: among these alkylthio radicals, there are preferably chosen from those mentioned above, those which contain at most 4 carbon atoms

The carboxy radical or radicals of the products of formula (I) can be salified or esterified by the various groups known to a person skilled in the art among which there can be mentioned, for example:

among salification compounds, mineral bases such as, for example, an equivalent of sodium, potassium, lithium, calcium, magnesium or ammonium or organic bases such as, for example, methylamine, propylamine, trimethylamine, diethylamine, triethylamine, N,N-dimethylethanolamine, tris (hydroxymethyl) amino methane, ethanolamine, pyridine, picoline, dicyclohexylamine, morpholine, benzylamine, procaine, lysine, arginine, histidine, N-methyl-glucamine,

among the esterification compounds, the alkyl radicals in order to form alkoxy carbonyl groups such as, for example, methoxycarbonyl, ethoxycarbonyl, tert-butoxycarbonyl or benzyloxycarbonyl, these alkyl radicals being able to be substituted by radicals chosen for example from halogen atoms, the hydroxyl, alkoxy, acyl, acyloxy, alkylthio, amino or aryl radicals such as, for example, in the chloromethyl, hydroxypropyl, methoxymethyl, propionyloxymethyl, methylthiomethyl, dimethylaminoethyl, benzyl or phenethyl groups.

The addition salts with mineral or organic acids of the products of formula (I) can be, for example, the salts formed with hydrochloric, hydrobromic, hydroiodic, nitric, sulphuric, phosphoric, propionic, acetic, trifluoroacetic, formic, benzoic, maleic, fumaric, succinic, tartaric, citric, oxalic, glyoxylic, aspartic, ascorbic acids, alkylmonosulphonic acids such as for example methanesulphonic acid, ethanesulphonic acid, propanesulphonic acid, alkyldisulphonic acids such as for example methanedisulphonic acid, alpha, beta-ethanedisulphonic acid, arylmonosulphonic acids such as benzenesulphonic acid and aryldisulphonic acids.

It should be remembered that stereoisomerism can be defined in its broadest sense as the isomerism of compounds having the same structural formulae, but the different groups of which are arranged differently in space, such as in particular in monosubstituted cyclohexanes the substituent of which can be in the axial or equatorial position, and the different possible rotational configurations of ethane derivatives. However, another type of stereoisomerism exists, due to the different spatial arrangements of fixed substituents, either on the double bonds, or on the rings, which is often called geometric isomerism or cis-trans isomerism. The term stereoisomers is used in the present Application in its widest sense and therefore relates to all of the compounds indicated above.

Therefore a subject of the present invention is the products of formula (I) as defined above in which R2, R3 and R4 have the meanings indicated above and R1 represents a cyclohexyl or heterocyclic, monocyclic or bicyclic phenyl radical containing 5 to 10 members, saturated or unsaturated, containing one or more identical or different heteroatoms chosen from O, N, NH or S and can contain a —C(O) member, the phenyl, cyclohexyl and heterocyclic radicals as defined above for R1, being optionally substituted by one or more radicals chosen from the halogen atoms; the following radicals: hydroxyl; cyclohexyl, cyano; nitro; free, salified or esterified carboxy; tetrazolyl; —NH2, —NH(alk), —N(alk)(alk); SO2-NH—CO—NHR5 in which R5 represents an alkyl or phenyl radical; phenyl; CF3; OCF3; alkyl, alkoxy and phenoxy themselves optionally substituted by the pyrazolinyl radical itself optionally substituted by one or more radicals chosen from the alkyl radicals and the halogen atoms, it being understood that in the above radicals, the alkyl and alkoxy radicals are linear or branched and contain at most 6 carbon atoms,

said products of formula (I) being in all possible racemic, enantiomeric and diastereoisomeric isomer forms, as well as the addition salts with mineral and organic acids or with mineral and organic bases of said products of formula (I). A more particular subject of the present invention is the products of formula (I) as defined above in which R2, R3 and R4 have the meanings indicated above and R1 represents a phenyl, cyclohexyl, pyrazolinyl, pyridyl, furyl, thienyl, isoxazolyl, isoquinolyl or quinolyl radical,

these radicals being optionally substituted by one or more radicals chosen from the halogen atoms; the following radicals: cyclohexyl; cyano; nitro; hydroxyl; free, salified or esterified carboxy; tetrazolyl; —NH2, —NH(alkyl), —N(alkyl)(alkyl); SO2-NH—CO—NHR5 in which R5 represents an alkyl or phenyl radical; phenyl; alkyl, alkoxy or phenoxy; CF3; OCF3; pyrazolinyl itself optionally substituted by one or more radicals chosen from the alkyl radicals and the halogen atoms,

it being understood that in the above radicals, the alkyl and alkoxy radicals are linear or branched and contain at most 4 carbon atoms,

said products of formula (I) being in all possible racemic, enantiomeric and diastereoisomeric isomer forms, as well as the addition salts with mineral and organic acids or with mineral and organic bases of said products of formula (I). A more particular subject of the present invention is the products of formula (I) as defined above, in which R1 has the meaning indicated above, R2 and R3 are such that one represents the hydrogen atom and the other represents a piperidinyl radical optionally substituted by a hydroxyl radical on a carbon-containing member and an alkyl radical on the nitrogen atom and R4 represents a hydrogen atom,

said products of formula (I) being in all possible racemic, enantiomeric and diastereoisomeric isomer forms, as well as the addition salts with mineral and organic acids or with mineral and organic bases of said products of formula (I). A more particular subject of the present invention is also the products of formula (I) as defined above, corresponding to the following formulae:

2-(2-chloro-4-fluorophenyl)-5,7-dihydroxy-8-[(3S,4R)-3-hydroxy-1-methyl-4-piperidinyl]-4H-benzopyran-4-one trifluoroacetate

2-(2,5-dichloro-3-thienyl)-5,7-dihydroxy-8-[(3S,4R)-3-hydroxy-1-methyl-4-piperidinyl]-4H-benzopyran-4-one trifluoroacetate

5,7-dihydroxy-8-[(3S,4R)-3-hydroxy-1-methyl-4-piperidinyl]-2-(5-methyl-3-isoxazolyl)-4H-benzopyran-4-one trifluoroacetate

5,7-dihydroxy-8-[(3S,4R)-3-hydroxy-1-methyl-4-piperidinyl]-2-[5-(trifluoromethyl)-1-phenyl-1H-pyrazol-4-yl]-4H-benzopyran-4-one trifluoroacetate

5,7-dihydroxy-6-[(3S,4R)-3-hydroxy-1-methyl-4-piperidinyl]-2-[3-(phenoxy)-phenyl]-4H-benzopyran-4-one trifluoroacetate

A subject of the present invention is also a process for the preparation of the products of formula (I), as defined above, characterized in that the compound of formula (II):

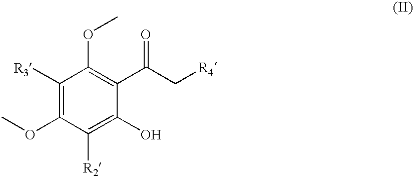

in which R2′, R3′ and R4′ have the meanings indicated above for R2, R3 and R4 respectively, in which the optional reactive functions are optionally protected by protective groups is subjected to a reaction with a compound of formula (III):

R1′COX (III)

in which R1′ has the meaning indicated above for R1, in which the optional reactive functions are optionally protected by protective groups and X represents a halogen atom or an alkoxy radical containing at most 6 carbon atoms, in order to obtain the product of formula (IV):

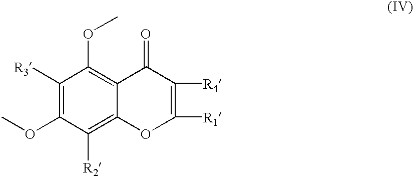

in which R1′, R2′, R3′ and R4′ have the meanings indicated above,

which products of formula (IV) are subjected to a deprotection reaction of the hydroxyl radicals in order to obtain a product of formula (I′):

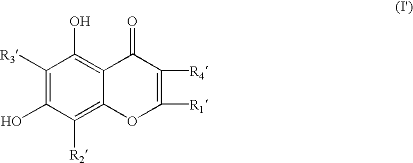

in which R1′, R2′, R3′ and R4′ have the meanings indicated above,

which products of formula (I′) can be products of formula (I) and which, in order to obtain some or other products of formula (I), can be subjected, if desired and if necessary, to one or more of the following conversion reactions, in any order:

a) an esterification reaction of an acid function,

b) a saponification reaction of the ester function to an acid function,

c) an oxidation reaction of the alkylthio group to a corresponding sulphoxide or sulphone,

d) a conversion reaction of the ketone function to an oxime function,

e) a reduction reaction of the free or esterified carboxy function to an alcohol function,

f) a conversion reaction of the alkoxy function to a hydroxyl function, or also of the hydroxyl function to an alkoxy function,

g) an oxidation reaction of the alcohol function to an aldehyde, acid or ketone function,

h) a conversion reaction of the nitrile radical to a tetrazolyl,

i) an elimination reaction of the protective groups which can be carried by the protected reactive functions,

j) a salification reaction by a mineral or organic acid or by a base in order to obtain the corresponding salt,

k) a resolution reaction of the racemic forms to resolved products,

said products of formula (I) thus obtained being in all possible racemic, enantiomeric and diastereoisomeric isomer forms.

It can be noted that such conversion reactions of substituents to other substituents can also be carried out on the starting products as well as on the intermediates as defined above before continuing the synthesis according to the reactions indicated in the process described above. In particular a subject of the present invention is a process for the deprotection of the hydroxyl functions of a product of formula (IV) as defined above:

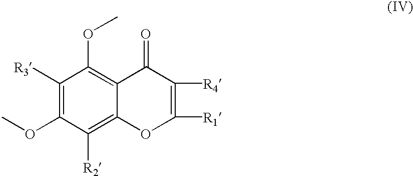

in which R1′, R2′, R3′ and R4′ have the meanings indicated below,

characterized in that the product of formula (IV) is subjected either to the action of hydroiodic acid supported by a resin in an anhydrous polar solvent,

or to the action of a salt of hydroiodic acid such as HI-Py in an anhydrous polar solvent,

or to the action of concentrated hydroiodic acid,

in order to obtain a product of formula (I′):

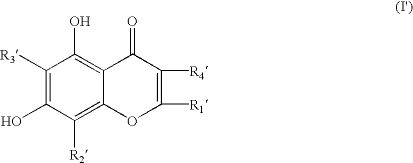

in which R1′, R2′, R3′ and R4′ have the meanings indicated above,

which products of formula (I′) can be the products of formula (I) and which, in order to obtain products or other products of formula (I), if desired and if necessary, can be subjected to one or more of the following conversion reactions, in any order:

a) an esterification reaction of the acid function,

b) a saponification reaction of the ester function to an acid function,

c) an oxidation reaction of the alkylthio group to a corresponding sulphoxide or sulphone,

d) a conversion reaction of the ketone function to an oxime function,

e) a reduction reaction of the free or esterified carboxy function to an alcohol function,

f) a conversion reaction of the alkoxy function to a hydroxyl function, or also the hydroxyl function to an alkoxy function,

g) an oxidation reaction of the alcohol function to an aldehyde, acid or ketone function,

h) a conversion reaction of the nitrile radical to a tetrazolyl,

i) an elimination reaction of the protective groups which can be carried by the protected reactive functions,

j) a salification reaction by a mineral or organic acid or by a base in order to obtain the corresponding salt,

k) a resolution reaction of the racemic forms to resolved products,

said products of formula (I) thus obtained being in all possible racemic, enantiomeric and diastereoisomeric isomer forms.

In the process indicated above, the product of formula (IV) is subjected to the action of hydroiodic acid supported by a resin such as for example Reillex-pyridine TM-402 polymer (poly 4-vinylpyridine) or also poly-2-vinylpyridine.

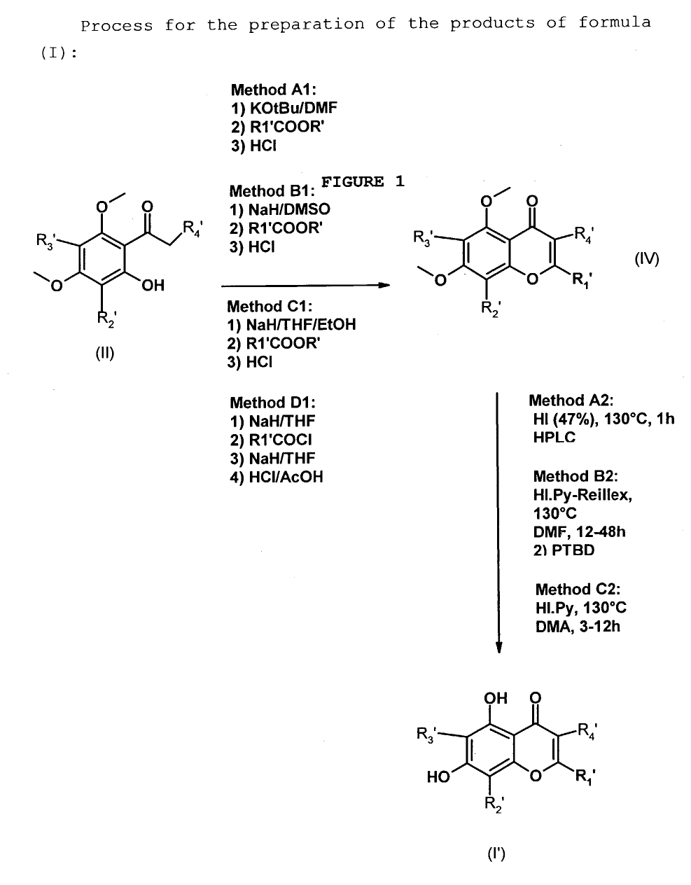

Under preferred conditions for implementing the invention, the process described above can be carried out as indicated in the diagram of FIG. 1 described hereafter: such a process can thus be carried out in the following fashion: The reaction of the product of formula (II) with a product of formula (III) in order to produce a product of formula (IV) can be carried out in particular according to four different methods called A1, B1, C1 and D1:

The first three use the product of formula (III) in which X represents an alkoxy radical and can be carried out in the following fashion:

for A1 the product of formula (II) is firstly subjected 1) to the action of KOtBu (potassium terbutylate) in a solvent such as DMF, then 2) to the action of the compound of formula (III) and finally 3) to the action of concentrated hydrochloric acid.

for B1 the product of formula (II) is firstly subjected 1) to the action of a base such as sodium hydride NaH in a solvent such as DMSO or DMF, then 2) to the action of the compound of formula (III) and finally 3) to the action of concentrated hydrochloric acid.

for C1 the product of formula (II) is firstly subjected 1) to the action of a base such as sodium hydride NaH in a solvent such as THF and in the presence of ethanol in a catalytic quantity (2 drops) and dibenzo-18-C-6 crown ether, then 2) to the action of the compound of formula (III) and finally 3) to the action of concentrated hydrochloric acid.

The fourth method D1 uses the product of formula (III) in which X represents a halogen atom and can be carried out in the following fashion:

the product of formula (II) is firstly subjected 1) to the action of a base such as sodium hydride NaH in a solvent such as THF or also DMF, then 2) to the action of the compound of formula (III) in which X represents a halogen atom then 3) again to the action of a base such as sodium hydride NaH in a solvent such as THF or also DMF and finally 4) to the action of concentrated hydrochloric acid in acetic acid (in the proportion of 1:10).

The product of formula (IV) thus obtained can be subjected to a deprotection reaction of the hydroxyl radicals in order to produce a product of formula (I′) in particular according to three different methods called A2, B2 and C2:

for A2 the product of formula (IV) is subjected to the action of concentrated hydroiodic acid at 130° C. for one hour

for B2 the product of formula (IV) is subjected, at 130° C. in a solvent such as DMF or also DMA for 12 to 48 hours, to the action of hydroiodic acid supported by a resin such as for example Reillex-pyridine TM-402 resin prepared as indicated in the experimental part,

for C2 the product of formula (IV) is subjected to the action of a salt of hydroiodic acid such as HI-Py (pyridine hydroiodide) at 130° C. in a solvent such as DMA or DMF for 3 to 12 hours.

The products of formula (IV) can therefore constitute the products of formula (I) in which the two hydroxyl functions are protected and which therefore produce after reaction according to A2, B2 or C2 a product of formula (I) as defined above.

The products of formula (IV) can also constitute the products of formula (I) in which the two hydroxyl functions are protected but also the other optionally reactive functions can be protected: the products of formula (IV), after deprotection of the hydroxyl radicals after reaction according to A2, B2 or C2, can then constitute the products of formula (I′) in which the optional reactive functions can be protected.

According to the values of R1′, R2′, R3′ and R4′, the products of formula (I′) constitute or do not constitute the products of formula (I) and can produce the products of formula (I), or be converted to other products of formula (I) by being subjected to one or more of reactions a) to k) indicated above.

Thus the various reactive functions which can be carried by certain reaction compounds defined above can, if necessary, be protected: it is for example the hydroxyl, acyl, free carboxy or also amino and monoalkylamino radicals which can be protected by the appropriate protective groups.

The following, non-exhaustive, list of examples of protection of the reactive functions can be mentioned:

the hydroxyl groups can be protected for example by the alkyl radicals such as tert-butyl, trimethylsilyl, tert-butyldimethylsilyl, methoxymethyl, tetrahydropyrannyl, benzyl or acetyl,

the amino groups can be protected for example by the acetyl, trityl, benzyl, tert-butoxycarbonyl, benzyloxycarbonyl, phthalimido radicals or other radicals known in peptide chemistry,

the acyl groups such as the formyl group can be protected for example in the form of cyclic or non cyclic ketals or thioketals such as dimethyl or diethylketal or ethylene dioxyketal, or diethylthioketal or ethylenedithioketal,

the acid functions of the products described above can, if desired, be amidified by a primary or secondary amine for example in methylene chloride in the presence, for example, of 1-ethyl-3-(dimethylaminopropyl) carbodiimide hydrochloride at ambient temperature:

the acid functions can be protected for example in the form of esters formed with easily-cleavable esters such as benzyl or terbutyl esters or esters known in peptide chemistry.

The reactions to which the products of formulae (IV) and (I′) as defined above can be subjected, if desired or if necessary, can be carried out, for example, as indicated hereafter.

a) The products described above can, if desired, be subjected, on the optional carboxy functions, to esterification reactions which can be carried out according to the usual methods known to a person skilled in the art.

b) The optional conversions of the ester functions to acid function of the products described above can be, if desired, carried out under the usual conditions known to a person skilled in the art in particular by acid or alkaline hydrolysis for example by soda or potash in an alcoholic medium such as, for example, in methanol or also by hydrochloric or sulphuric acid.

c) The optional alkylthio groups of the products described above can be, if desired, converted to the corresponding sulphoxide or sulphone functions under the usual conditions known to a person skilled in the art such as for example by peracids such as for example peracetic acid or metachloroperbenzoic acid or also by ozone, oxone, sodium periodate in a solvent such as for example methylene chloride or dioxan at ambient temperature.

Obtaining the sulphoxide function can be encouraged by an equimolar mixture of the product containing an alkylthio group and the reagent such as in particular a peracid. Obtaining the sulphone function can be encouraged by a mixture of the product containing an alkylthio group with an excess of the reagent such as in particular a peracid.

d) The conversion reaction of a ketone function to oxime can be carried out under the usual conditions known to a person skilled in the art, such as in particular an action in the presence of an optionally O-substituted hydroxylamine in an alcohol such as for example ethanol, at ambient temperature or while heating.

e) The optional free or esterified carboxy functions of the products described above can be, if desired, reduced to the alcohol function by methods known to a person skilled in the art: the optional esterified carboxy functions can be, if desired, reduced to the alcohol function by methods known to a person skilled in the art and in particular by lithium and aluminium hydride in a solvent such as for example tetrahydrofuran or also dioxane or ethyl ether.

The optional free carboxy functions of the products described above can be, if desired, reduced to the alcohol function in particular by boron hydride.

f) The optional alkoxy functions such as in particular methoxy of the products described above can be, if desired, converted to the hydroxyl function under the usual conditions known to a person skilled in the art and in particular according to the conditions described hereafter in the experimental part.

g) The optional alcohol functions of the products described above can be, if desired, converted to the aldehyde or acid function by oxidation under the usual conditions known to a person skilled in the art such as for example by the action of manganese oxide in order to obtain the aldehydes or Jones reagent for accessing acids.

h) The optional nitrile functions of the products described above can be, if desired, converted to tetrazolyl under the usual conditions known to a person skilled in the art such as for example by the cycloaddition of a metallic azide such as for example sodium azide or a trialkyltin azide on the nitrile function as indicated in the method described in the article referenced as follows:

J. Organometallic Chemistry., 33, 337 (1971) KOZIMA S. et al.

It can be noted that the conversion reaction of a carbamate to urea and in particular of a sulphonylcarbamate to sulphonylurea, can be carried out for example under reflux of a solvent such as for example toluene in the presence of the suitable amine.

It is understood that the reactions described above can be carried out as indicated or also, if appropriate, according to other usual methods known to a person skilled in the art.

i) The elimination of the protective groups such as for example those indicated above can be carried out under the usual conditions known to a person skilled in the art in particular by an acid hydrolysis carried out with an acid such as hydrochloric, benzene sulphonic or paratoluene sulphonic, formic or trifluoroacetic acid or also by a catalytic hydrogenation.

The phthalimido group can be eliminated by hydrazine. A list of the different protective groups which can be used will be found for example in the Patent BF 2 499 995.

j) The products described above can, if desired, be the subject of salification reactions for example by a mineral or organic acid or by a mineral or organic base according to the usual methods known to a person skilled in the art.

k) The optional optically active forms of the products described above can be prepared by resolution of the racemics according to the usual methods known to a person skilled in the art.

Illustrations of such reactions defined above are given in the preparation of the examples described hereafter. The products of formula (I) as defined above as well as their addition salts with acids have useful pharmacological properties.

The products of the present invention as defined above, have kinase inhibitory properties of great selectivity. The cdk's play a central role in the initiation, the development and the completion of the events of the cell cycle and thus, the inhibitory molecules of cdk are capable of limiting undesirable cell proliferations such as those observed in cancers, psoriasis, fungal growth, parasites (animals, protists): such inhibitory molecules of cdk are thus also capable of intervening in the regulation of neurodegenerative diseases such as Alzheimer's disease. Kinases which are particularly sensitive to the inhibitory effects of the derivatives of the present invention are in particular cdk1, cdk2, cdk4, cdk5 and cdk7.

The products of the present invention are therefore endowed with antimitotic properties.

The products of the present invention have in addition to their specific inhibitory properties of kinases, useful cellular effects such as antiproliferative properties and in particular effects on apoptosis.

It is known from the work described in the literature such as in WO 97/20842, that relationships exist between the cell cycle and apoptosis. Among the routes leading to apoptosis, certain are dependent on kinases.

The products of the present invention are in particular useful for tumour therapy.

The products of the invention can also therefore increase the therapeutic effects of the anti-tumor agents which are currently used.

The products of formula (I) of the present invention therefore have quite particularly antimitotic and anti-neurodegenerative properties.

These properties justify their use in therapeutics and a particular subject of the invention is as medicaments, the products of formula (I) as defined above, said products of formula (I) being in all possible racemic, enantiomeric and diastereoisomeric isomer forms, as well as the addition salts with pharmaceutically acceptable mineral and organic acids or with mineral and organic bases of said products of formula (I).

A more particular subject of the invention is also as medicaments, the products of formula (I) as defined above, as well as the addition salts with pharmaceutically acceptable mineral and organic acids or with mineral and organic bases of said products of formula (I).

A quite particular subject of the invention is as medicaments, the products described hereafter in the examples and in particular the products of formula (I) as defined above, corresponding to the following formulae:

2-(2-chloro-4-fluorophenyl)-5,7-dihydroxy-8-[(3S,4R)-3-hydroxy-1-methyl-4-piperidinyl]-4H-benzopyran-4-one trifluoroacetate

2-(2,5-dichloro-3-thienyl)-5,7-dihydroxy-8-[(3S,4R)-3-hydroxy-1-methyl-4-piperidinyl]-4H-benzopyran-4-one trifluoroacetate

5,7-dihydroxy-8-[(3S,4R)-3-hydroxy-1-methyl-4-piperidinyl]-2-(5-methyl-3-isoxazolyl)-4H-benzopyran-4-one trifluoroacetate

5,7-dihydroxy-8-[(3S,4R)-3-hydroxy-1-methyl-4-piperidinyl]-2-[5-(trifluoromethyl)-1-phenyl-1H-pyrazol-4-yl]-4H-benzopyran-4-one trifluoroacetate

5,7-dihydroxy-6-[(3S,4R)-3-hydroxy-1-methyl-4-piperidinyl]-2-[3-(phenoxy)-phenyl]-4H-benzopyran-4-one trifluoroacetate,

as well as the addition salts with pharmaceutically acceptable mineral and organic acids or with mineral and organic bases of said products of formula (I).

The medicaments, which are a subject of the invention, are of use, for example, as antimitotics, in the chemotherapy of cancers, or also in the treatment of psoriasis, parasitosis such as those due to protists or to fungi or also in the treatment of Alzheimer's disease or in the treatment of neuronal apoptosis.

The invention extends to the pharmaceutical compositions containing at least one of the medicaments defined above as active ingredient.

Such pharmaceutical compositions according to the present invention can also, if appropriate, contain the active ingredients of other antimitotic medicaments such as in particular those based on taxol, cisplatin, DNA intercalating agents and others.

These pharmaceutical compositions can be administered by buccal route, by parenteral route or by local route as a topical application on the skin and mucous membranes or by injection by intravenous or intramuscular route.

These compositions can be solids or liquids and be presented in all the pharmaceutical forms commonly used in human medicine, such as for example, plain or sugar-coated tablets, pills, lozenges, gelatin capsules, drops, granules, injectable preparations, ointments, creams or gels; they are prepared according to the usual methods. The active ingredient can be incorporated with the excipients usually used in these pharmaceutical compositions such as talc, gum arabic, lactose, starch, magnesium stearate, cocoa butter, aqueous or non-aqueous vehicles, fatty substances of animal or vegetable origin, paraffin derivatives, glycols, various wetting, dispersing or emulsifying agents, preservatives. The dose administered is variable according to the product used, the patient treated and the illness in question and can be, for example, from 0.05 to 5 g per day in an adult, or preferably from 0.1 to 2 g per day.

Among the starting products of formula (II), some are known and can be obtained commercially or can be prepared according to the usual methods known to a person skilled in the art. Certain starting products of formula (II) can be prepared as indicated in the Patent Application number FR9807677: there can be mentioned in particular the starting product of formula (II) in which R3 and R4 both represent a hydrogen atom and R2 represents the piperidinyl radical substituted by a hydroxyl radical on one carbon-containing member and methyl on the nitrogen atom. Such a product is in particular 1-[2-hydroxy-4,6-dimethoxy-3-[(3S,4R)-3-hydroxy-1-methyl-4-piperidinyl]-phenyl]-ethanone also called (−)-cis-acetoflocino-piperidinol or also Acetoflocinopiperidol prepared as indicated in the Patent Application number FR 9807677.

Among the starting products of formula (III), certain are known and can be obtained commercially or can be prepared according to the usual methods known to a person skilled in the art.

Among the commercial starting products of formula (III) in which X represents an alkoxy radical, there can be mentioned for example, the following products of formula (III):

methyl 2-chloro-4-fluorobenzoate,

methyl 2-5-bis(2,2,2-trifluoroethoxy)benzoate,

dimethyl 2,5-dichloroterephthalate

methyl 2,5-dimethylfuran-3-carboxylate.

Among the commercial starting products of formula (III) in which X represents a halogen atom, there can be mentioned for example, the following products of formula (III):

3-(trifluoromethoxy)benzoyl chloride

3-(trifluoromethyl)-4-(fluoro)benzoyl chloride.

Certain starting products can also in particular be prepared from commercial products for example by subjecting them to one or more of the reactions described above in a) to k), carried out under the conditions also described above.

The experimental part given hereafter gives examples of such starting products.

Finally a subject of the present invention is as new industrial products, the products of formula (IV) as defined above.

Thus a particular subject of the invention is the pharmaceutical compositions containing, at least one of the medicaments as defined above, as active ingredient

A quite particular subject of the invention is the pharmaceutical compositions as defined above characterized in that they are used as antimitotic medicaments, in particular for the chemotherapy of cancers or also in the treatment of psoriasis, parasitosis such as those due to fungi or to protists or Alzheimer's disease.

A quite particular subject of the invention is also the pharmaceutical compositions as defined above characterized in that they are used as antineurodegenerative medicaments in particular neuronal anti-apoptosis.

A particular subject of the present invention is the use of the products of formula (I) as defined above for the preparation of medicaments intended for the prevention or treatment of diseases linked to a deregulation of the secretion and/or of the activity of protein tyrosine kinases.

A particular subject of the present invention is the use of the products of formula (I) as defined above for the preparation of medicaments intended for the chemotherapy of cancers, for the treatment of psoriasis, parasitosis such as those due to fungi or to protists, for the treatment of Alzheimer's disease or for the treatment of neurodegenerative affections in particular neuronal apoptosis.

The following examples illustrate the invention without however limiting it.

1) Preparation of the Pyridine hydroiodide reagent (Py-HI) 500 cm3 of ethyl anhydrous ether, 10 cm3 of pyridine (m=79.10) (d =0.978, 123.6 mmol, 1.2 eq.), 13.5 cm3 of HI (hydroiodic acid)(57%) (M=127.91) (d=1.7, 102.3 mmol) are introduced dropwise at AT.

At the end of the introduction of HI, the ethereal phase is eliminated by decantation, the pasty precipitate is rinsed with ether then recrystallized from 30 ml of absolute EtOH under reflux.

After filtration and drying under vacuum the expected product is obtained in the form of white crystals. Weight: 16.87 g

2) Preparation of the Reillex-Py-HI reagent

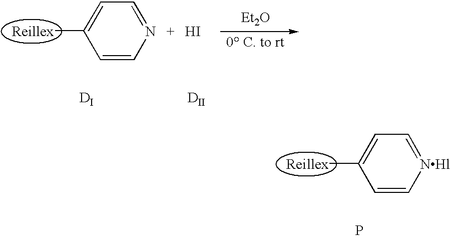

The compounds used are DI and DII defined as follows:

DI: Reillex TM 402 polymer cross-linked with 2% DVB-4-pyridine (˜7 mmol/g).

DII: hydroiodic acid HI(57%) d 1.70 (˜7.6M) 18.42 ml (˜140 mmol) of DII is added at 0° C. under argon to 350 ml of ether Et2O containing 10 g (˜70 mmol) of DI. The temperature slowly rises to ambient temperature and the reaction medium is left overnight.

The solution obtained is then filtered, the resin is then washed with ether (3×), ethanol (1×), then again with ether (1×). After passage under vacuum, 21 g of expected product is obtained i.e. Reillex-Py.HI: the product obtained is such that 11 g of HI (hydroiodic acid) are fixed on DI and the molarity of the product obtained is therefore: (11/128)/21=0.004 M/g=4 mmol/g.

### EXAMPLE 1

### 2-(2-chloro-4-fluorophenyl)-5,7-dihydroxy-8-[(3S,4R)-3-hydroxy-1-methyl-4-piperidinyl]-4H-benzopyran-4-one trifluoroacetate

Stage a): 2-(2-chloro-4-fluoropheny]-5,7-dimethoxy-8-[(3S,4R)-3-hydroxy-1-methyl-4-piperidinyl]-4H-benzopyran-4-one

1) Condensation: 1-(2-chloro-4-fluorophenyl)-3-[2-hydroxy-4,6-dimethoxy-3-[(3S,4R)-3-hydroxy-1-methyl-4-piperidinyl] phenyl]-1,3-propanedione

380 mg of NaH at 50% in oil (M=24) (8 mmol-4 eq.) and 9.5 cm3 of DMSO/NK20, 618 mg of 1-[2-hydroxy-4,6-dimethoxy-3-[(3S,4R)-3-hydroxy-1-methyl-4-piperidinyl]-phenyl]-ethanone also called Acetoflocinopiperidol (2 mmol) prepared as indicated in the Patent Application number FR 9807677 are introduced under N2. Agitation is carried out for 1 hour at ambient temperature then 1.13 g of methyl 2-chloro-4-fluoro-benzoate (M=188.59) (6 mmol-3 eq.) is introduced and the reaction medium is maintained under agitation for 20 hours at ambient temperature.

2) Cyclization: 2-(2-chloro-4-fluoropheny)-5,7-dimethoxy-8-[(3S,4R)-3-hydroxy-1-methyl-4-piperidinyl]-4H-benzopyran-4-one

3 cm3 of 36% HCl (36 mmol-18 eq.) is added to the reaction medium obtained above in 1), and the medium is then taken to 50° C. for 2 hours. The reaction medium returns to ambient temperature, followed by pouring into ice-cooled water, then the pH is rendered basic by adding concentrated soda, followed by extracting three times with 50 cm3 of CH2Cl2/MeOH: 9/1, washing with water, drying over anhydrous MgSO4 and bringing to dryness under vacuum. 1.5 g of a brown oil is obtained which is chromatographed on silica 0.04-0.06 mm in CH2Cl2/MeOH: 90/10. The expected product is isolated in the form of yellow solid of 90 mg in weight.

Analyses:

¹H NMR Spectrum: CDCl3 δ (ppm) 1.57(bd), 3(masked): 2H; 2 11(t,b), 3.05(masked): 2H; 2.28(m), 3.05(m): 2H; 2.37(s): CH3-N; 3.50(ddd): 1H; 3.95(s,b): 1H(equatorial); 3.97(s), 4.00(s): 6H; 6.43(s): 1H; 6.46(s): 1H; 7.14(ddd): 1H; 7.30(dd): 1H; 7.60(dd): 1H.

Stage b): 2-(2-chloro-4-fluorophenyl)-5,7-dihydroxy-8-[(3S,4R)-3-hydroxy-1-methyl-4-piperidinyl]-4H-benzopyran-4-one trifluoroacetate

90 mg of dimethoxy (0.2 mmol) obtained in Stage a) above, 1.8 cm3 of anhydrous DMF and 550 mg of Reillex-pyridine-HI (≈4 mmol/g)(22 moll-11 eq.) obtained as indicated in Preparation 2 above are introduced under N2 into a 30 cm3 flask with a reflux condenser. The reaction medium is taken to 130° C. for 24 hours and to 150° C. for 4 hours. Then it is returned to ambient temperature, diluted with CH3CN, filtered on Iéna, rinsed with CH3CN then 1.5 g of PTBD resin (i.e. 1,5,7 Triazabicyclo-[4,4,0]dec-5-ene) (2.6 mmol/g) (4 mmol-20 eq.) is added. Agitation is carried out for 20 hours at ambient temperature, followed by filtering on Iéna, washing with CH3CN then releasing with 20 cm3 of CH3CN/TFA: 95/05 under agitation. The reaction medium is filtered on Iéna, rinsed with CH3CN/TFA: 95/05 then taken to dryness under vacuum. 160 mg of an amorphous orange product is recovered which is taken up in 5 cm3 of the HPLC eluent described hereafter, followed by filtering on a microporous filter-0.45 μm then injecting three times into peparative HPLC: Kromasil C18-10 μm—500×22 mm—λ=225 nm—15 ml/min with: CH3CN/H2O/TFA: 35/65/0.1 as eluent. The appropriate fractions are combined, concentrated under vacuum then lyophilized. The expected product is isolated in the form of a yellow solid of 42 mg in weight.

Analyses:

¹H NMR spectrum: DMSO D⁶ δ (ppm) 1.83(bd), 2.98(m): 2H; 3.08(m), 3.36(masked): 2H; 3.29(m): 2H; 3.44(m): 1H(axial); 2.72(d): CH3-N; 4.06(bs): 1H(equatorial); 6.00(bs): OH; 6.39(s): 1H; 6.60(s): 1H; 7.47(ddd): 1H; 7.75(dd): 1H; 7.92(dd): 1H; 9.30(bs): N⁺H; 11.30(bs), 12.97(s): 2 OH.

### EXAMPLE 2

### 2-(2,5-dichloro-3-thienyl)-5,7-dihydroxy-8-[(3S,4R)-3-hydroxy-1-methyl-4-piperidinyl]-4H-benzopyran-4-one trifluoroacetate

Stage a): 2-(2,5-dichloro-3-thienyl)-5,7-dimethoxy-8-[(3S,4R)-3-hydroxy-1-methyl-4-piperidinyl]-4H-benzopyran-4-one

1) Condensation: 1-(2,5-dichloro-3-thienyl)-3-[2-hydroxy-4,6-dimethoxy-3-[(3S,4R)-3-hydroxy-1-methyl-4-piperidinyl] phenyl]-1,3-propanedione

310 mg of NaH at 50% in oil (M=24) (6.5 mmol-4 eq.), 10 cm3 of anhydrous THF, 500 mg of Acetoflocinopiperidol (1.6 mmol) by fractions, 2 drops of EtOH and 12 mg of Dibenzo-18-Crown-6 (M=360.41) (0.03 mmol-2% molar) are introduced, under N2, into a 30 cm3 flask. Agitation is carried out for 5 minutes at ambient temperature, then 1.02 g of methyl 2,5-dichloro-3-thiophenecarboxylate (M=211.07) (4.8 mmol-3eq.) is introduced and the reaction medium is taken under reflux for 4 hours 30 minutes.

2) Cyclization: 2-(2,5-dichloro-3-thienyl)-5,7-dimethoxy-8-[(3S,4R)-3-hydroxy-1-methyl-4-piperidinyl]-4H-benzopyran-4-one

10 cm3 of 36% HCl (116 mmol-72 eq.) is added to the reaction medium obtained above in 1), and maintained under reflux for 2 hours. After returning to ambient temperature, the reaction medium is poured into 100 cm3 of NaOH(2N), followed by extracting with 2×100 cm3 of CH2Cl2/MeOH: 8/2, washing with a saturated solution of NaCl, drying over anhydrous Na2SO4 and bringing to dryness under vacuum. 0.76 g of a black resin is obtained which is chromatographed on silica 0.04-0.06 mm in CH2Cl2/MeOH: 80/20. In this way the expected product of 147 mg in weight is isolated.

Analyses:

IR spectrum: Absorption OH/NH region —C═O: 1644 cm-1; aromatic conjugated system: 1594, 1567, 1538, 1493 cm-1. ¹H NMR spectrum: DMSO D⁶ δ (ppm) 1.51(bd), 3.03(masked): 2H; 2.11(t,b), 2.95(masked): 2H; 2.27(masked), 2.94(masked): 2H; 2.28(s): CH3-N; 3.36(masked): 1H(axial); 3.76(s,b): 1H(equatorial); 3.89(s), 3.93(s): 6H; 4.49(bs): OH; 6.66(s): 1H; 6.51(s): 1H; 7.76(s): 1H.

Stage b): 2-(2,5-dichloro-3-thienyl)-5,7-dihydroxy-8-[(3S,4R)-3-hydroxy-1-methyl-4-piperidinyl]-4H-benzopyran-4-one trifluoroacetate

115 mg of the dimethoxy obtained in Stage a) above (0.24 mmol), 2.4 cm3 of DMA and 506 mg of Pyridine-HI (M=207.01) (2.4 mmol-10 eq.) obtained as indicated in Preparation 1 above are introduced, under N2, into a 20 cm3 flask with a reflux condenser. The reaction medium is taken to 130° C. for 18 hours, followed by returning to ambient temperature, diluting with CH3CN, then adding 1.9 g of PTBD resin (2.6 mmol/g) (4.9 mmol-20 eq.) Agitation is carried out for 1 hour 30 minutes at ambient temperature, followed by filtering on Iéna, washing with CH3CN then releasing with 10 cm3 of CH3CN/TFA: 95/05 under agitation. Then filtration is carried out on Iéna, followed by rinsing with CH3CN/TFA: 95/05 then bringing to dryness under vacuum. 217 mg of a brown resin is recovered which is taken up in 10 cm3 of MeOH and which is saponified with 1 cm3 of NaOH (2N) (2 mmol-8 eq.). The reaction medium is then taken to 60° C. for 5 hours 30 minutes and returned to ambient temperature overnight. After bringing to dryness under vacuum, 350 mg of a brown solid is obtained which is taken up in 6.5 cm3 of the HPLC eluent described hereafter, filtered on a microporous filter-0.45 μm then injected 4 times into preparative HPLC: Kromasil C18—10 μm—500×22 mm—λ=225 nm with: CH3CN/H2O/TFA: 40/60/0.1—15 ml/min as eluent. The appropriate fractions are collected which are concentrated under vacuum then lyophilized. The expected product is isolated in the form of a yellow solid of 28 mg in weight.

Analyses:

¹H NMR spectrum: DMSO D⁶ δ (ppm) 1.83(d), 3.03(m): 2H; 2.79(s): CH3-N; 3.13(m), 3.42(masked): 2H; 3.26(masked), 3.36(masked): 2H; 3.54(d): 1H(axial); 4.13(s): 1H(equatorial); 6.35(s): 1H; 6.73(s): 1H; 7.62(s): 1H; 9.37(bs), 11.31(bs), 12.9(s): 3H (mobiles).

### EXAMPLE 3

### 5,7-dihydroxy-8-[(3S,4R)-3-hydroxy-1-methyl-4-piperidinyl]-2-(5-methyl-3-isoxazolyl)-4H-benzopyran-4-one, trifluoroacetate

Stage a): 5,7-dimethoxy-8-[(3S,4R)-3-hydroxy-1-methyl-4-piperidinyl]-2-(5-methyl-3-isoxazolyl)-4H-benzopyran-4-one

1) Condensation: 1-[2-hydroxy-4,6-dimethoxy-3-[(3S,4R)-3-hydroxy-1-methyl-4-piperidinyl]phenyl]-3-(5-methyl-3-isoxazolyl)1,3-propanedione

310 mg of NaH at 50% in oil (M=24) (6.5 mmol-4 eq.), 10 cm3 of anhydrous THF, 500 mg of Acetoflocinopiperidol (1.6 mmol) by fractions, 2 drops of EtOH and 12 mg of Dibenzo-18-Crown-6 (M=360.41) (0.03 mmol-2% molar) is introduced, under N2, into a 30 cm3 flask. Agitation is carried out for 30 minutes at ambient temperature, then 684 mg of methyl 5-methyl-3-isoxazolecarboxylate (M=141.13) (4.8 mmol-3 eq.) is introduced. The reaction medium is maintained under agitation at ambient temperature, for 18 hours.

2) Cyclization: 5,7-dimethoxy-8-[(3S,4R)-3-hydroxy-1-methyl-4-piperidinyl]-2-(5-methyl-3-isoxazolyl)-4H-benzopyran-4-one 10 cm3 of 36% HCl (116 mmol-72 eq.) are added to the reaction medium obtained in 1) and the medium is then taken to 50° C. for 2 hours. The reaction medium is returned to ambient temperature, poured into 100 cm3 of NaOH(2N), extracted with 2×200 cm3 of CH2Cl2/MeOH: 8/2, followed by washing with a saturated solution of NaCl, drying over anhydrous Na2SO4 and bringing to dryness under vacuum. 650 mg of a beige solid is obtained which is chromatographed on silica 0.04-0.06 mm in CH2Cl2/MeOH: 90/10 then CH2Cl2/MeOH/NH4OH: 90/10/1. The expected product is isolated in the form of beige solid of 418 mg in weight.

Analyses:

IR spectrum Absorption OH/NH region —C═O: 1658 cm-1; Conjugated system+aromatic: 1628, 1595, 1568 cm-1. ¹H NMR spectrum: DMSO D⁶ δ (ppm) 1.47(bd), 3.14(m): 2H; 1.97 (t,b), 2.93(bd): 2H; 2.20(masked), 2.88(bd): 2H; 2.23(s): CH3-N; 3.29(bd): 1H (axial); 3.94(masked): 1H(OH); 3.69(s,b): 1H (equatorial); 3.90(s), 3.94(s): 6H; 6.66(s): 1H; 6.72(s), 6.93(s): 2H; 2.51 cm asked): 3H.

Stage b): 5,7-dihydroxy-8-[(3S,4R)-3-hydroxy-1-methyl-4-piperidinyl]-2-(5-methyl-3-isoxazolyl)-4H-benzopyran-4-one trifluoroacetate

90 mg of the dimethoxy obtained in Stage a) above (0.22 mmol), 2.5 cm3 of anhydrous DMF and 538 mg of Reillex-pyridine-HI (≈4 mmol/g) (2.15 mmol-10 eq.) is introduced, under N2, into a 30 cm3 flask with a reflux condenser. The reaction medium is taken to 130° C. for 18 hours 45 minutes and to 150° C. for 5 hours 30 minutes. Then the reaction medium is returned to ambient temperature, diluted with CH3CN, filtered on Iéna, rinsed with CH3CN then 1.7 g of PTBD resin (2.6 mmol/g) (4.4 mmol-20 eq.) is added. Agitation is carried out for 1 hour at ambient temperature, followed by filtering on Iéna, washing with CH3CN then releasing with 20 cm3 of CH3CN/TFA: 95/05 under agitation, filtering on Iéna, rinsing with CH3CN/TFA: 95/05 then bringing to dryness under vacuum. 205 mg of a yellow varnish is recovered which is taken up in 3 cm3 of HPLC eluent described hereafter. Filtration is carried out on a microporous filter-0.45 μm then injection three times into preparative HPLC: Kromasil C18—10 μm—500×22 mm—λ=225 nm—15 ml/min. with CH3CN/H2O/TFA: 30/70/0.1 as eluent. The appropriate fractions are combined which are concentrated under vacuum then lyophilized. The expected product is isolated in the form of cream solid of 43 mg in weight.

Analyses:

Spectrum ¹H NMR: DMSO D⁶ δ (ppm) 1.85(bd), 3.35(masked): 2H; 2.55(s): 3H; 2.80(s): N—CH3; 3.14(m), 3.47(m): 2H; 3.36(masked): 2H; 3.47(m): 1H (axial); 4.07(bs): 1H(equatorial); 6.39(s): 1H; 6.94, 7.00: 2H; 5.77(bs), 9.40(bs), 11.32(bs), 12.87(s): 4H (mobile).

### EXAMPLE 4

### 5,7-dihydroxy-8-[(3S,4R)-3-hydroxy-1-methyl-4-piperidinyl]-2-[5-(trifluoromethyl)1-phenyl-1H-pyrazol-4-yl]-4H-benzopyran-4-one trifluoroacetate

Stage a): 5,7-dimethoxy-8-[(3S,4R)-3-hydroxy-1-methyl-4-piperidinyl]-2-[5-(trifluoromethyl)1-phenyl-1H-pyrazol-4-yl]-4H-benzopyran-4-one

1) Condensation: 1-[2-hydroxy-4,6-dimethoxy-3-[(3S,4R)-3-hydroxy-1-methyl-4-piperidinyl]phenyl]-3-[5-(trifluoromethyl) 1-phenyl-1H-pyrazol-4-yl]-1,3-propanedione

310 mg of NaH at 50% in oil (M=24) (6.5 mmol-4 eq.), 10 cm3 of anhydrous THF, 500 mg of Acetoflocinopiperidol (1.6 mmol) by fractions, 2 drops of EtOH and 12 mg of Dibenzo-18-Crown-6 (M=360.41) (0.03 mmol-2% molar) are introduced, under N2, into a dry 50 cm3 flask. Agitation is carried out for 30 minutes at ambient temperature, then 1.3 g of methyl 5-(trifluoromethyl)1-phenyl-1H-pyrazole-4-carboxylate (M=270.21) (4.8 mmol-3 eq.) is introduced. The reaction medium is taken to reflux for 2 hours 30 minutes.

2) Cyclization: 5,7-dimethoxy-8-[(3S,4R)-3-hydroxy-1-methyl-4-piperidinyl]-2-[5-(trifluoromethyl)1-phenyl-1H-pyrazol-4-yl]-4H-benzopyran-4-one

10 cm3 of 36% HCl (116 mmol-72 eq.) is added to the reaction medium obtained above in 1), and the medium is then taken to 80° C. for 1 hour. After returning to ambient temperature, the reaction medium is poured into 100 cm3 of NaOH (2N), followed by extracting with 2×200 cm3 of CH2Cl2/MeOH: 8/2, washing with a saturated solution of NaCl, drying over anhydrous Na2SO4 and bringing to dryness under vacuum. 3.72 g of an orange oil is obtained which is chromatographed on silica 0.04-0.06 mm in CH2Cl2/MeOH/NH4OH: 90/10/0.2 then 90/10/1. The expected product is isolated in the form of a beige foam of 454 mg in weight.

Analyses:

IR spectrum: Absorption OH/NH region —C═O: 1656 cm-1; Conjugated system+aromatic: 1627, 1596, 1570, 1500 cm-1.

¹H NMR spectrum:. DMSO D⁶ δ (ppm) 1.47(bd), 3.03(m): 2H; 1.97(t,b), 2.87(masked): 2H; 2.87(masked), 2.18(masked): 2H; 2.21(s): CH3-N; 3.29(masked): 1H(axial); 3.71(s,b): 1H(equatorial); 3.90(s), 3.94(s): 6H; 4.32(bd): 1H(OH); 6.31(s): 1H; 6.67(s): 1H; 7.63(bs): 5H; 8.55(s): 1H.

Stage b): 5,7-dihydroxy-8-[(3S,4R)-3-hydroxy-1-methyl-4-piperidinyl]-2-[5-(trifluoromethyl)1-phenyl-1H-pyrazol-4-yl]-4H-benzopyran-4-one trifluoroacetate

117 mg of the dimethoxy obtained above in Stage a) (0.22 mmol), 2.5 cm3 of anhydrous DMF, 538 mg of Reillex-pyridine-HI (≈4 mmol/g) (2.15 mmol-10 eq.) are introduced, under N2, into a 30 cm3 flask with a reflux condenser. The reaction medium is taken to 130° C. for 37 hours and to 150° C. for 5 hours. Then it is returned to ambient temperature, diluted with CH3CN, filtered on Iéna, rinsed with CH3CN then 1.7 g of PTBD resin (2.6 mmol/g) (4.4 mmol-20 eq.) is added. Agitation is carried out for 4 hours 30 minutes at ambient temperature, followed by filtering on Iéna, washing with CH3CN then releasing with 20 cm3 of CH3CN/TFA: 95/05 under agitation, filtering on Iéna, rinsing with CH3CN/TFA: 95/05 then bringing to dryness under vacuum. 195 mg of a yellow resin is recovered which is taken up in 5 cm3 of HPLC eluent described hereafter, filtering on a microporous filter-0.45 μm then injecting twice into preparative HPLC: Kromasil C18—10 μm—500×22 mm λ=225 nm—15 ml/min. with: CH3CN/H2O/TFA: 40/60/0.1 as eluent. The appropriate fractions are combined, concentrated under vacuum then lyophilized. The expected product is isolated in the form of a slightly yellow solid of 83 mg in weight.

Analyses:

¹H NMR spectrum: DMSO D⁶ δ (ppm) 1.79(bd), 3.08(m): 2H; 2.79(d): CH3-N; 3.10(m), 3.45(m): 2H; 3.28(bt), 3.38(m): 2H; 3.51(m): 1H(axial); 4.08(bs): 1H(equatorial); 6.39(s): 1H; 6.55(s): 1H; 6.27(bs): 1H (OH); 7.64(bs): 5H; 8.54(s): 1H; 9.43(bs): 1H NH⁺; 11.34(bs), 12.94(s): 2H(OH).

### EXAMPLE 5

### trifluoroacetate of methyl 4-[5,7-dihydroxy-8-[(3S,4R)-3-hydroxy-1-methyl-4-piperidinyl]-4-oxo-4H-benzopyran-2-yl]-cyclohexanecarboxylate

Stage a): methyl 4-[5,7-dimethoxy-8-[(3S,4R)-3-hydroxy-1-methyl-4-piperidinyl]-4-oxo-4H-benzopyran-2-yl]cyclohexanecarboxylate

1) Condensation: methyl 4-[3-[2-hydroxy-4,6-dimethoxy-3-[(3S,4R)-3-hydroxy-1-methyl-4-piperidinyl]phenyl]1,3-dioxopropyl]-cyclohexanecarboxylate

390 mg of NaH at 50% in oil M=24 (8 mmol-4 eq.), 10 cm3 of DMSO/NK20 are introduced, under N2, into a 30 cm3 flask and 618 mg of Acetoflocinopiperidol (2 mmol) is added. Agitation is carried out for 1 hour at ambient temperature, then 1.1 cm3 (600 mg) of dimethyl 1,4-cyclohexanedicarboxylate (M=200.24) (3 mmol-3 eq.) is introduced. The reaction medium is maintained under agitation for 20 hours. The reaction is treated by pouring into ice-cooled water, followed by adjusting to pH 7 by adding 2N HCl, extracting with a CH2Cl2/MeOH: 90/10 mixture, washing with water, drying over MgSO4, filtering and bringing to dryness under vacuum with a rotary evaporator. The crude product obtained of 3.58 g in weight, is chromatographed on silica 0.04-0.06 mm in CH2Cl2/MeOH: 80/20. The fractions containing the expected product are isolated which are brought to dryness and dried under vacuum: in this way the expected product is obtained in the form of resin of 300 mg in weight.

2) Cyclization: methyl 4-[5,7-dimethoxy-8-[(3S,4R)-3-hydroxy-1-methyl-4-piperidinyl]-4-oxo-4H-benzopyran-2-yl] cyclohexanecarboxylate

300 mg of above product in 1) (0.62 mmol), 1.5 cm3 of anhydrous DMSO and 0.063 cm3 of 36% conc. HCl (0.75 mmol-1.2 eq) are placed in a flask under agitation. The reaction medium is taken to 50° C. for 1 hour. The reaction is finished, and the medium is poured into ice-cooled water, brought to pH 9-10 by adding conc. soda (1.5 cm3), followed by extracting three times with 20 cm3 of CH2Cl2/MeOH: 90/10, washing with water, drying over MgSO4, filtering and bringing to dryness.

Chromatography is carried out on a silica column 0.04-0.06 mm in CH2Cl2/MeOH: 80/20 eluent and the expected product is isolated in the form of a yellow solid of 145 mg in weight: the TRANS product is in the majority.

Analyses:

¹H NMR spectrum: CDCl3 δ (ppm) 2.50(tt): 1H; 2.35(tt): 1H; 1.45(m), 2.11(m), 2.14(m), 1.59(m): 8H; 2.48(s): N—CH3; 2.41, 3.22: 2H; 2.25, 3.22: 2H; 1.66, 3.22: 2H; 3.92(b): 1H(eq); 3.41(ddd): 1H(axial); 2.80(b): OH; 3.70(s), 3.94(s), 3.96(s): 9H; 6.00(s): 1H; 6.40(s): 1H.

Stage b): trifluoroacetate of methyl 4-[5,7-dihydroxy-8-[(3S,4R)-3-hydroxy-1-methyl-4-piperidinyl]-4-oxo-4H-benzopyran-2-yl]-cyclohexanecarboxylate

A) Deblocking: trifluoroacetate of 4-[5,7-dihydroxy-8-[(3S,4R)-3-(formyloxy)-1-methyl-4-piperidinyl]-4-oxo-4H-benzopyran-2-yl]-cyclohexanecarboxylic acid

120 mg of the dimethoxy obtained above in Stage a) (0.26 mmol), 2.6 cm3 of anhydrous DMA and 540 mg of Pyridine HI M=207.01 (2.6 mmol-10 eq.) are introduced, under N2, into a 30 cm3 flask with a reflux condenser. The reaction medium is taken to 130° C. for 20 hours.

The reaction medium is then returned to ambient temperature, followed by diluting with CH3CN, then adding 1.9 g of PTBD resin (2.6 mmol/g) (5.2 mmol-20 eq.). Agitation is carried out for 2 hours at ambient temperature, followed by filtering on Iéna, washing with CH3CN then releasing with 10 cm3 of CH3CN/TFA: 95/05 under agitation, filtering on Iéna, rinsing with CH3CN/TFA: 95/05 then bringing to dryness under vacuum. In this way 160 mg of expected product is obtained.

B) Esterification: trifluoroacetate of methyl 4-[5,7-dihydroxy-8-[(3S,4R)-3-hydroxy-1-methyl-4-piperidinyl]-4-oxo-4H-benzopyran-2-yl]-cyclohexanecarboxylate

160 mg of the product obtained in A) above (0.26 mmol estimated), 1.6 cm3 of methanol and 0.25 cm3 of ClSiMe3 (M=108.64 d=0.856) are placed in a flask under agitation and under argon. The reaction medium is left at 50° C. for 48 hours, followed by bringing to dryness under vacuum with a rotary evaporator. In this way 150 mg of product is obtained which is purified by HPLC: the 150 mg of product thus obtained are dissolved in 1 cm3 of a solution of CF3CO2Na at 0.5 M/l to which 2 cm3 of HPLC eluent: CH3CN/H2O/TFA: 35/65/0.1 is added. Filtration is carried out on a microporous filter-0.45 μm then injection three times into preparative HPLC: Kromasil C18—10 μm—500×22 mm—λ=225 nm—15 ml/min. The appropriate fractions are combined, concentrated under vacuum then lyophilized. In this way the expected product is isolated in the form of solid of 48 mg in weight.

Analyses:

¹H NMR spectrum: DMSO D⁶ δ (ppm) 2.79: N—CH3; 3.35: 2H; 5.74: 1H(OH); 4.00(bs): 1H(eq); 3.40(masked): 1H(axial); 3.13, 1.79: 2H; 3.47, 3.12: 2H; 3.63(s): 3H; 2.59, 1.96, 1.65, 1.48, 2.03, 2.44: 10 H; 6.14: 1H; 6.30: 1H; 9.34(bs), 11.1: 3H.

### EXAMPLE 6

### 2-(4-cyclohexylphenyl)-5,7-dihydroxy-8-[(3S,4R)-3-hydroxy-1-methyl-4-piperidinyl]-4H-benzopyran-4-one trifluoroacetate

Stage a): 2-(4-cyclohexylphenyl)-5,7-dimethoxy-8-[(3S,4R)-3-hydroxy-1-methyl-4-piperidinyl]-4H-benzopyran-4-one

1) Condensation: 1-(4-cyclohexylphenyl)-3-[2-hydroxy-4,6-dimethoxy-3-[(3S,4R)-3-hydroxy-1-methyl-4-piperidinyl] phenyl]-1,3-propanedione

288 mg of NaH at 50% in oil (M=24) (4.5 mmol-4 eq), 4.5 cm3 of DMSO/NK20 and 0.464 mg of Acetoflocinopiperidol (1.5 mmol) are introduced, under N2, into a 30 cm3 flask. Agitation is carried out for 1 hour at ambient temperature, then 0.982 mg of methyl 4-cyclohexyl-benzoate (M=218.3) (4.5 mmol-3 eq.) is introduced and the reaction medium is maintained under agitation for 20 hours.

2) Cyclization: 2-(4-cyclohexylphenyl)-5,7-dimethoxy-8-[(3S,4R)-3-hydroxy-1-methyl-4-piperidinyl]-4H-benzopyran-4-one

3.25 cm3 of 36% HCl (39 mmol-25 eq) is added to the reaction medium obtained above in 1), and the whole is then taken to 50° C. for 3 hours. The reaction medium is returned to ambient temperature, treated by diluting with 5 cm3 of water and 3.5 cm3 of 12 N NaOH is added followed by extracting 3 times with 20 cm3 of CH2Cl2/MeOH: 9/1, washing with water, drying over anhydrous Na2SO4 and bringing to dryness under vacuum. 1.4 g of a brown oil is obtained which is chromatographed on silica 0.04-0.06 mm in CH2Cl2/MeOH: 90/10. The expected product is isolated in the form of a yellow solid of 300 mg in weight.

Analyses:

¹H NMR spectrum: CDCl3 δ (ppm) 1.2, 1.95: 10 H; 2.58(tt): 1H; 2.41(s): N—CH3; 3.96(s), 3.99(s): 6H; 2.33(bd), 3.15: 2H; 4.09(bs): 1H(eq); 3.61(ddd): 1H(axial); 3.12, 1.64: 2H; 2.15, 3.09: 2H; 6.44(s): 1H; 6.64(s): 1H; 7.37, 7.74: 4H.

Stage b): 2-(4-cyclohexylphenyl)-5,7-dihydroxy-8-[(3S,4R)-3-hydroxy-1-methyl-4-piperidinyl]-4H-benzopyran-4-one trifluoroacetate

113 mg of the dimethoxy obtained in Stage a) above (0.24 mmol) and 1.3 cm3 of HI (57%) are introduced, under N2, into a 20 cm3 flask with a reflux condenser. The reaction medium is taken to 130° C. for 1 hour 30 minutes, then returned to ambient temperature, followed by diluting with 3 cm3 of MeOH, adjusting to pH 10 by adding 2 cm3 of a 6M/l soda solution, followed by filtering on a microporous filter-0.45 μm then injecting 4 times into preparative HPLC: Kromasil C18—10 μm—500×22 mm—λ=225 nm—15 ml/min. with: MeOH/H2O/TFA: 75/25/0.1 as eluent. The appropriate fractions are combined and concentrated under vacuum then lyophilized. In this way the expected product is isolated in the form of a yellow solid of 41 mg in weight.

Analyses:

¹H NMR spectrum: DMSO D⁶ δ (ppm) 1.28, 1.73: 2H; 1.82, 1.41: 4H; 1.45, 1.82: 4H; 2.62(m): 1H; 2.82(s): CH3-N; 1.83, 3.20: 2H; 3.20, 3.47: 2H; 3.40: 2H; 3.54(bd): 1H(axial); 4.10(bs): 1H(equatorial); 6.35(s): 1H; 6.89(s): 1H; 8.02: 2H; 7.45: 2H; 5.98(b): 1H(OH); 9.44(b), 11.17(b), 13.20(b): 3H mobile.

### EXAMPLE 7

### 4-[5,7-dihydroxy-8-[(3S,4R)-3-hydroxy-1-methyl-4-piperidinyl]-4-oxo-4H-benzopyran-2-yl]-benzonitrile trifluoroacetate

Stage a): 4-[5,7-dimethoxy-8-[(3S,4R)-3-hydroxy-1-methyl-4-piperidinyl]-4-oxo-4H-benzopyran-2-yl]-benzonitrile

1) Condensation: 4-[3-[2-hydroxy-4,6-dimethoxy-3-[(3S,4R) 3-hydroxy-1-methyl-4-piperidinyl]phenyl]-1,3-dioxopropyl]-benzonitrile.

392 mg of NaH at 50% in oil (M=24) (8 mmol-4 eq.), 10 cm3 of DMSO/NK20 and 618 mg of Acetoflocinopiperidol (2 mmol) are introduced, under N2, into a 20 cm3 flask. Agitation is carried out for 1 hour at ambient temperature, then 966 mg of methyl 4-cyanobenzoate (M=161.16) (6 mmol-3 eq.) is introduced. The reaction medium is maintained under agitation for 20 hours at ambient temperature.

2) Cyclization: 4-[5,7-dimethoxy-8-[(3S,4R)-3-hydroxy-1-methyl-4-piperidinyl]-4-oxo-4H-benzopyran-2-yl]-benzonitrile 3 cm3 of 36% HCl (36 mmol-18 eq.) is added to the reaction medium obtained above in 1), and the whole is then taken to 50° C. for 1 hour 30 minutes. The reaction medium is returned to ambient temperature. The expected product precipitates in the medium. The precipitate is filtered, followed by washing with ether and drying under vacuum. The product obtained is in the form of the hydrochloride in the form of a yellow solid of 1.4 g in weight.

The product obtained is released from its salt by treatment with 1N soda and extracted with a CH2Cl2/MeOH: 90/10 mixture, three times 20 cm3. In this way 600 mg of expected product is obtained.

Analyses:

IR spectrum:—Absorption OH/NH region —CN: 2228 cm-1; C═O: 1650 cm-1; System conjugated+aromatic: 1619, 1594, 1568, 1557, 1497 cm-1 ¹H NMR spectrum: CDCl3 δ (ppm) 7.93, 7.82AA′BB′: 4H; 6.69(s): 1H; 6.47(s): 1H; 4.01(s), 3.98(s): 6H; 4.0: 1H; 2.39(s): CH3-N; 3.10(m), 1.61: 2H; 3.09(m), 2.11(t): 2H; 3.15(m), 3.28: 2H; 1.80: 1H(OH); 3.51(m): 1H.

Stage b): 4-[5,7-dihydroxy-8-[(3S,4R)-3-hydroxy-1-methyl-4-piperidinyl]-4-oxo-4H-benzopyran-2-yl]-benzonitrile trifluoroacetate

112 mg of the dimethoxy obtained above in Stage a) (0.26 mmol), 2.6 cm3 of anhydrous DMF and 732 mg of Reillex-pyridine-HI (≈4 mmol/g) (2.86 mmol-10 eq.) obtained in preparation 2) above are introduced, under N2, into a 30 cm3 flask with a reflux condenser. The reaction medium is taken to 130° C. for 40 hours, then returned to ambient temperature, followed by diluting with MeOH, filtering on Iéna, rinsing with MeOH then bringing to dryness under vacuum with a rotary evaporator. Weight. of crude product obtained: 500 mg. This product is solubilized in 6 cm3 of a 6M/l soda solution of MeOH (pH of the medium˜10), filtered on a microporous filter-0.45 μm then injected 6 times into preparative HPLC: Kromasil C18—10 μm—500×22 mm—λ=225 nm—15 ml/min with: MeOH/H2O/TFA: 55/45/0.1 as eluent. The appropriate fractions are combined and concentrated under vacuum then lyophilized. In this way the expected product is isolated in the form of a yellow solid of 75 mg in weight.

Analyses:

¹H NMR spectrum: DMSO D⁶ δ (ppm) 3.16, 1.81(bd): 2H; 2.83: CH3-N; 3.16, 3.47(masked): 2H; 3.40(masked): 2H; 3.53(bt): 1H(eq); 4.09(bs): 1H(axial); 6.39(s): 1H; 7.09(s): 1H; 8.30, 8.05AA′BB′: 4H; 5.97, 11.31, 13.03, 9.42: mobile 4H's.

### EXAMPLE 8

### 5,7-dihydroxy-8-[(3S,4R)-3-hydroxy-1-methyl-4-piperidinyl]-2-[4-(1H-tetrazol-5-yl)-phenyl]-4H-benzopyran-4-one trifluoroacetate

Stage a): 5,7-dimethoxy-8-[(3S,4R)-3-hydroxy-1-methyl-4-piperidinyl]-2-[4-(1H-tetrazol-5-yl)-phenyl]-4H-benzopyran-4-one

200 mg of the nitrile product obtained in Stage a) of Example 7 (M=420.47) (0.47 mmol), 4 cm3 of dry toluene and 180 mg of trimethyltin azide (M=205.81) (0.87 mmol-1.85 eq) are introduced, under N2, into a 30 cm3 flask provided with a condenser. The reaction medium is taken to reflux for 43 hours then cooled down and the precipitate formed is filtered, followed by washing and drying under vacuum overnight. 300 mg of crude product is obtained. The crude product is chromatographed on silica 0.04-0.06 in CH2Cl2/MeOH/NH4OH: 78/20/02. In this way the expected product is isolated in the form of a solid of 174 mg in weight.

Analyses:

¹H NMR spectrum: DMSO D⁶ δ (ppm) 3.38(masked): 2H; 4.06(bs): 1H(equatorial); 3.60(bd): 1H(axial); 3.33(masked), 1.83(bd): 2H; 3.27, 3.38(masked): 2H; 2.81(s): N—CH3; 6.70(s): 1H; 6.67(s): 1H; 3.91(s): 3H; 3.94(s): 3H; 5.53(bs): 1H(OH); 8.06, 8.20 AA′BB′: 4H.

Stage b): 5,7-dihydroxy-8-[(3S,4R)-3-hydroxy-1-methyl-4-piperidinyl]-2-[4-(1H-tetrazol-5-yl)-phenyl]-4H-benzopyran-4-one trifluoroacetate

120 mg of the dimethoxy obtained in Stage a) above (0.26 mmol), 2.4 cm3 of anhydrous DMF and 700 mg of Reillex-pyridine-HI (≈4 mmol/g) (2.86 mmol-11 eq.) obtained in Preparation 2) above are introduced, under N2, into a 30 cm3 flask with a reflux condenser. The reaction medium is taken to 130° C. for 40 hours, then returned to ambient temperature, filtered on Iéna, rinsed with MeOH then with DMF. The solid product obtained is washed with water and dried under vacuum. 209 mg of crude product is obtained. This crude product is solubilized in approximately 4 cm3 of HPLC eluent: CH3CN/H2O/TFA: 25/75/0.1. The medium is adjusted to pH 10 by adding 2N soda (0.45 cm3), followed by filtering on a microporous filter-0.45 μm then injecting 5 times into preparative HPLC: Kromasil C18—10 μm—500×22 mm—λ=225 nm—15 ml/min—.

The appropriate fractions are combined and concentrated under vacuum then lyophilized. In this way the expected product is isolated in the form of a yellow solid of 88 mg in weight.

Analyses:

¹H NMR spectrum: DMSO D⁶ δ (ppm) 3.43(bs): 2H; 4.11(bs): 1H(eq); 3.57(bd): 1H(ax) 3.20(masked), 1.84(bd): 2H; 3.21(masked), 3.49(bd): 2H; 2.84(s): N—CH3; 7.08(s): 1H; 6.38(s): 1H; 8.29, 8.35 AA′BB′: 4H; 5.99(bs): 1H(OH); 9.36(bs), 11.25(bs); 13.11(s): 3H(mobile).

### EXAMPLE 9

### 5,7-dihydroxy-8-[(3S,4R)-3-hydroxy-1-methyl-4-piperidinyl]-2-[3-(phenoxy)-phenyl]-4H-benzopyran-4-one trifluoroacetate

Stage a): 5,7-dimethoxy-8-[(3S,4R)-3-hydroxy-1-methyl-4-piperidinyl]-2-[3-(phenoxy)phenyl]-4H-benzopyran-4-one

1) Condensation: 1-[2-hydroxy-4,6-dimethoxy-3-[(3S,4R)-3-hydroxy-1-methyl-4-piperidinyl]phenyl]-3-[3-(phenoxy)phenyl]-1,3-propanedione

196 mg of NaH at 50% in oil (M=24) (4 mmol-4 eq.), 5 cm3 of DMSO/NK20 and 309 mg of Acetoflocinopiperidol (1 mmol) are introduced, under N2, into a 10 cm3 flask. Agitation is carried out for 1 hour at ambient temperature, then 685 mg of methyl 3-(phenoxy)-benzoate (M=228.2) (3 mmol-3 eq.) is introduced. The reaction medium is maintained under agitation for 20 hours. The medium is treated with 3.5 cm3 of 1N HCl until the pH=6-7, followed by extracting with 3 times 10 cm3 of methylene chloride, then washing with water, drying over MgSO4, filtering and bringing to dryness under vacuum. In this way 1.5 g of crude product is obtained.

2) Cyclization: 5,7-dimethoxy-8-[(3S,4R)-3-hydroxy-1-methyl-4-piperidinyl]-2-[3-(phenoxy)phenyl]-4H-benzopyran-4-one 474 mg of crude product (0.32 mmoles) obtained above in 1) and 0.5 cm3 of 36% HCl (6 mmol-18 eq.) are placed in a 10 cm3 flask. The reaction medium is then taken to 50° C. for 2 hours, returned to ambient temperature, diluted with 5 cm3 of water, 0.55 cm3 of 32% NaOH is added, followed by extracting 3 times with 10 cm3 of CH2Cl2/MeOH: 9/1, washing with water, drying over anhydrous Na2SO4 and bringing to dryness under vacuum. 150 mg of a brown oil is obtained which is chromatographed on silica 0.04-0.06 mm in CH2Cl2/MeOH: 95/05. In this way the expected product is obtained in the form of a yellow solid of 220 mg in weight.

Analyses:

IR spectrum: Absorption OH/NH region —C═O: 1646 cm-1; conjugated system+aromatic: 1620, 1597, 1578, 1489 cm-1. ¹H NMR spectrum: CDCl3 δ (ppm) 2.33(s): N—CH3; 3.95(s), 3.98(s): 6H; 6.44(s): 1H; 6.62(s): 1H; 1.57(m), 3.10(m): 2H; 2.10(m), 2.98(m): 2H; 2.03(m), 3.02(m): 2H; 3.48(ddd): 1H(ax); 3.93(b): 1H(eq); 7.06: 2H; 7.16: 1H; 7.38: 2H; 7.19(ddd): 1H; 7.45(dd): 1H; 7.50(t): 1H; 7.56(ddd): 1H.

Stage b): 5,7-dihydroxy-8-[(3S,4R)-3-hydroxy-1-methyl-4-piperidinyl]-2-[3-(phenoxy)-phenyl]-4H-benzopyran-4-one trifluoroacetate

200 mg of the dimethoxy obtained in Stage a) above (0.41 mmol) and 3 cm3 of 47% HI(1604 mmol-40 eq.) are introduced, under N2, into a 30 cm3 flask with a reflux condenser and the whole is taken to 130° C. for 1 hour 30 minutes. The reaction is stopped and brought to dryness under vacuum using a rotary evaporator. 340 mg of an amorphous orange product is recovered which is taken up in 6 cm3 of HPLC eluent described hereafter, followed by filtering on a microporous filter-0.45 μm then injecting 4 times into preparative HPLC: Kromasil C18—10 μm—500×22 mm—λ=225 nm—15 ml/min. with MeOH/H2O/TFA: 65/35/0.1 as eluent. The appropriate fractions are combined and concentrated under vacuum then lyophilized. The expected product is isolated in the form of a yellow solid of 25 mg in weight.

Analyses:

¹H NMR spectrum: DMSO D⁶ δ (ppm) 2.78(s): N—CH3; 3.23, 3.33: 2H; 4.07: 1H; 3.49: 1H; 3.14, 1.80: 2H; 3.14, 3.37: 2H; 5.99: 1H(OH); 6.93: 1H; 6.36: 1H; 13.11, 11.23: 2H(mobile); 7.68: 1H; 7.26: 1H; 7.69: 1H; 7.91: 1H; 7.11: 2H; 7.45: 2H; 7.21: 1H.

### EXAMPLE 10

### 5,7-dihydroxy-6-[(3S,4R)-3-hydroxy-1-methyl-4-piperidinyl]-2-[3-(phenoxy)-phenyl]-4H-benzopyran-4-one trifluoroacetate

In Stage b) of Example 9, a second product is isolated in the form of a yellow solid of 16 mg in weight.

Analyses:

¹H NMR spectrum: DMSO D⁶ δ (ppm) 2.74: N-Me; 3.29: 2H; 4.07: 1H; 3.40: 1H; 1.62, 3.05: 2H; 3.06, 3.36: 2H; 6.96: 1H; 6.45: 1H; 7.71: 1H; 7.18: 1H; 7.57: 1H; 7.84: 1H; 7.09: 2H; 7.44: 2H; 7.19: 1H; 13.58: 1H(mobile); 9.08(bs): 2H mobile.

### EXAMPLE 11

### 5,7-dihydroxy-8-[(3S,4R)-3-hydroxy-1-methyl-4-piperidinyl]-2-(3-nitrophenyl)-4H-benzopyran-4-one

Stage a): 1-[4,6-dimethoxy-3-[(3S,4R)-3[(ethoxycarbonyl)oxy]-1-methyl-4-piperidinyl]-2-hydroxy phenyl]-ethanone 19 ml of CH2Cl2 and 1.13 g of Acetoflocinopiperidol (3.65 mmol) are introduced, under N2, into a 30 cm3 flask, then 348 μl of ethyl chloroformate (C3H5ClO2, M=108.52, d=1.139, 3.65 mmol, 1.0 eq.) is introduced dropwise at −78° C. The reaction medium is left to return to ambient temperature slowly and left for 48 hours at this temperature. The reaction medium is diluted in 100 ml of CH2Cl2, followed by washing twice with H20 with 10% of NaHCO3 and once with H2O saturated in NaCl. After drying over MgSO4, filtration and evaporation, 1.23 g of expected product is obtained.

Analyses:

¹H NMR spectrum: CDCl3 δ (ppm) 1.19(t, J=7): 3H; 4.02(qd): 2H; 1.54(m), 3.10(m): 2H; 2.08(m), 3.01(m): 2H; 2.31(bs): N—CH3; 3.09(m), 2.32(masked): 2H; 4.90(b): 1H(eq); 3.35(ddd, J=2,5-4,0-13.0): 1H(ax); 2.61(s): 3H; 3.84(s), 3.89(s): 6H; 5.90(s): 1H; 14.34(s): 1H(OH). IR spectrum: OH in chelated form —C═O: 1733 cm-1; C═O+aromatic: 1612, 1598 (max), 1582(sh), 1503 cm-1(f).

Stage b): 5,7-dimethoxy-8-[(3S,4R)-3-[(ethoxycarbonyl)oxy]-1-methyl-4-piperidinyl]-2-(3-nitrophenyl)-4H-benzopyran-4-one 1.09 g of the product obtained in Stage a) above (2.87 mmol) and 15 cm3 of anhydrous THF are introduced, under N2, into a 60 cm3 flask, then 462 mg of tBuOK (M=112) (4.12 mmol-1.4 eq.) is introduced rapidly at 0° C.

After 1 hour at this temperature, 692 mg of 3-nitro-benzoyl chloride (M=185.57) (3.73 mmol-1.3 eq.) is introduced. The medium is left to return to AT and agitation is carried out for 12 hours. Then 398 mg of tBuOK (M=112) (3.45 mmol-1.2 eq.) is introduced at AT. The reaction medium is taken to 65° C. and left for 4 hours. Then 15 ml of AcOH and 1.5 ml of concentrated HCl are introduced and the reaction medium is taken to 65° C. for 4 hours, returned to ambient temperature, and the solvents are evaporated off under vacuum. The residues are taken up in 200 cm3 of CH2Cl2/MeOH: 9/1, washed with a saturated solution of NaHCO3, and H2O saturated in NaCl, followed by drying over anhydrous MgSO4 and bringing to dryness under vacuum. 3.7 g of a brown solid is obtained which is chromatographed on silica 0.04-0.06 mm in CH2Cl2/MeOH: 90:10. The expected product is isolated in the form of a yellow solid of 205 mg in weight.

Analyses:

¹H NMR spectrum: CDCl3 δ (ppm) 3.25, 2.49: 2H; 2.39(s): CH3-N; 3.12, 3.18: 2H; 2.73, 3.22: 2H; 3.63(td): 1H(ax); 3.96(s), 4.01(s): 6H; 1.06: 3H; 3.94: 2H; 5.0(bs): 1H(eq); 6.44(s): 1H; 6.72(s): 1H; 8.78(s): 1H; 8.38(dd): 1H; 8.40, 8.30: 1H; 7.78(t): 1H.

Stage c): 5,7-dimethoxy-8-[(3S,4R)-3-hydroxy-1-methyl-4-piperidinyl]-2-(3-nitrophenyl)-4H-benzopyran-4-one 200 mg of the product obtained in Stage b) above (0.34 mmol), 2.4 ml of ethanol and 0.4 ml of NaOH (2N) (0.8 mmol, 2; 35 eq) are introduced, under N2, into a 10 cm3 flask. The medium is agitated at AT for 2 hours and diluted in 4.8 ml of H2O and the ethanol is left to evaporate overnight. The precipitates are filtered on frit and washed with H2O (3×2 ml), and with ether (5 ml). After drying under vacuum, the expected product is obtained in the form of a yellow solid of 132 mg in weight.

Stage d): 5,7-dihydroxy-8-[(3S,4R)-3-hydroxy-1-methyl-4-piperidinyl]-2-(3-nitrophenyl)-4H-benzopyran-4-one 100 mg of the dimethoxy obtained in Stage c) above (0.23 mmol), 2.3 cm3 of anhydrous DMF and 624 mg of Reillex-pyridine-HI (≈4 mmol/g) (2.5 mmol-11 eq) obtained in Preparation 2) are introduced, under N2, into a 30 cm3 flask with a reflux condenser. The reaction medium is taken to 130° C. for 24 hours, then it is returned to ambient temperature, diluted with MeOH (5 ml), filtered on Iéna, rinsed with MeOH (2×5 ml). The methanol is evaporated off under vacuum, the residues are taken up in 2.0 ml of a solution of MeOH/H2O/CF3COONa (50/50, 0.5M), the pH of the medium of this mixture is adjusted to 8-9 with NaOH (N), followed by filtering on Iéna, washing with H2O (3×3 ml). After drying under vacuum, the expected product is isolated in the form of a yellow solid=80 mg.

Analyses:

¹H NMR spectrum: DMSO D⁶ δ (ppm) 1.52, 2.81: 2H; 2.84, 3.30: 2H; 2.96, 3.24: 2H; 2.62(s): CH3-N; 3.43: 1H; 4.08(bs): 1H; 5.78(s): 1H; 6.97(s): 1H; 7.86(t): 1H; 8.40, 8.46: 2H; 8.72: 1H; 12.76: 1H(OH).

### EXAMPLE 12

### 5,7-dihydroxy-2-[4-fluoro-3-(trifluoromethyl)phenyl]-8-[(3S,4R)-3-hydroxy-1-methyl-4-piperidinyl]-4H-benzopyran-4-one trifluoroacetate

Stage a): 5,7-dimethoxy-2-[4-fluoro-3(trifluoromethyl) phenyl]-8-[(3S,4R)-3-hydroxy-1-methyl-4-piperidinyl]-4H-benzopyran-4-one 52 mg of NaH at 50% in oil (M=24) (1.08 mmol-1.2 eq.) and 9.0 cm3 of anhydrous THF are introduced, under N2, into a 60 cm3 flask, then 344 mg of the product obtained in Stage a) of Example 11 (0.90 mmol) is introduced at −78° C. After 1 hour at this temperature, 225 μl of 4-fluoro-3-(trifluromethyl)-benzoyl chloride (M=226.56) (d=1.493, 1.50 mmol, 1.7 eq) is introduced. The medium is left to return to AT and agitation is carried out for 12 hours. Then 129 mg of NaH at 50% in oil (M=24) (2.69 mmol-3.0 eq.) is introduced at 0° C. The medium is left under agitation at AT for 72 hours. Then 9 ml of AcOH and 1 ml of concentrated HCl are introduced. The reaction medium is then taken to 65° C. for 3 hours, returned to ambient temperature and the solvents are evaporated off under vacuum. The residues are taken up in 100 cm3 of CH2Cl2/MeOH: 9/1, followed by washing with a saturated solution of NaHCO3, and H2O saturated in NaCl, drying over anhydrous MgSO4 and bringing to dryness under vacuum. 703 mg of an oil is obtained which is chromatographed on silica 0.04-0.06 mm in CH2Cl2/MeOH: 90/10. In this way the expected product is isolated in the form of a yellow solid=148 mg.

Analyses:

¹H NMR spectrum: CDCl3 δ (ppm) 2.60(m), 3.46(m): 2H; 4.17(bs): 1H; 3.52(m): 1H; 1.67(bd), 3.31(m): 2H; 3.29(m), 2.41(m): 2H; 3.48(bs): 6H; 6.42(bs), 6.52(s): 2H; 7.37(t): 1H; 8.16(bs): 1H; 7.94(bs): 1H.

Stage b): 5,7-dihydroxy-2-[4-fluoro-3-(trifluoromethyl)phenyl]-8-[(3S,4R)-3-hydroxy-1-methyl-4-piperidinyl]-4H-benzopyran-4-one trifluoroacetate 139 mg of the dimethoxy obtained in Stage a) above (0.29 mmol), 2.9 cm3 of anhydrous DMF and 725 mg of Reillex-pyridine-HI (≈4 mmol/g) (2.9 mmol-10 eq.) obtained as indicated in Preparation 2) above are introduced, under N2, into a 30 cm3 flask with a reflux condenser. The reaction medium is taken to 130° C. for 42 hours, then returned to ambient temperature, diluted with CH3CN (10 ml), filtered on Iéna, rinsed with CH3CN (2×10 ml) then 2.23 g of PTBD resin (2.6 mmol/g) (5.8 mmol-20 eq.) is added. Agitation is carried out for 1 hour at ambient temperature, followed by filtering on Iéna, washing with CH3CN (100 ml) then releasing with 20 cm3 of CH3CN/TFA: 95/05 under agitation, filtering on Iéna, rinsing with CH3CN/TFA: 95/05 (2×10 ml) then bringing to dryness under vacuum. 144 mg of an amorphous orange product is recovered which is taken up in 3 cm3 of HPLC eluent: CH3CN/H2O/TFA: 35/65/0.1, followed by filtering on a microporous filter-0.45 μm then injecting twice into preparative HPLC: Kromasil C18—10 μm -500×22 mm—λ=225 nm—15 ml/min. The appropriate fractions are combined and concentrated under vacuum then lyophilized. In this way the expected product is isolated in the form of a yellow solid of 76.2 mg in weight.

Analyses:

¹H NMR spectrum: DMSO D⁶ δ (ppm) 1.83, 3.09: 2H; 2.80(s): N—Me; 3.13, 3.41: 2H; 3.34: 2H; 3.53(bd), 4.11(bs): 1H(ax), 1H(eq); 6.32: 1H; 7.13: 1H; 7.47(t): 1H; 8.39(dd): 1H; 8.43(d): 1H.

### EXAMPLE 13

### 2-(2-chloro-3-pyridinyl)-5,7-dihydroxy-8-[(3S,4R)-3-hydroxy-1-methyl-4-piperidinyl]-4H-benzopyran-4-one trifluoroacetate

Stage a): (3S,4R)-4-[2-(2-chloro-3-pyridinyl)-5,7-dimethoxy-4-oxo-4H-benzopyran-8-yl]-1-methyl-3-piperidinyl 2-chloro-3-pyridinecarboxylate

586 mg of Acetoflocinopiperidol (DI) (1.89 mmol) and 19.0 cm3 of anhydrous THF are introduced, under N2, into a 60 cm3 flask. Then 400 mg of NaH at 50% in oil (M=24) (8.3 mmol-4.4 eq.) is introduced at 0° C. After 1 hour at this temperature, 1.70 g of 2-chloro-3-pyridinecarbonyl chloride (M=176.56) (5.67 mmol, 3 eq) is introduced. The reaction medium is left for 1 hour at 0° C., for 4 hours at AT, and for 12 hours at 65° C. The medium is left to return to AT and then 110 mg of NaH at 50% in oil (M=24) (2.27 mmol-1.2 eq.) is introduced. The medium is agitated for 1 hour at AT and for 4 hours at 65° C. Then 19 ml of AcOH and 1.9 ml of concentrated HCl are introduced and the reaction medium is taken to 80° C. for 12 hours, allowed to return to ambient temperature, and the solvents are evaporated off under vacuum. The residues are taken up in 150 cm3 of CH2Cl2/MeOH: 9/1, followed by washing twice with a solution of NaOH (1N) and H2O saturated in NaCl, drying over anhydrous MgSO4 and bringing to dryness under vacuum. 2.5 g of crude products are obtained which are chromatographed on silica 0.04-0.06 mm in CH2Cl2/MeOH/NH3.H2O: 90:10:01. The expected product is isolated in the form of a yellow solid of 315 mg in weight.

Analyses:

¹H NMR spectrum: CDCl3 δ (ppm) 3.23(bd), 2.37(bd): 2H; 3.07(bt), 1.79(bd): 2H; 3.05, 2.09(bt): 2H; 2.30(s): CH3-N; 3.62(td): 1H(ax); 5.39(bs): 1H(eq); 3.99(s), 3.85(s): 6H; 6.40(s): 1H; 6.50(s): 1H; 7.23(dd): 1H; 7.97(dd): 1H; 8.45(dd): 1H; 7.39(dd): 1H; 7.85(dd): 1H; 8.53(dd): 1H.

Stage b): 2-(2-chloro-3-pyridinyl)-5,7-dimethoxy-8-[(3S,4R)-3-hydroxy-1-methyl-4-piperidinyl]-4H-benzopyran-4-one

300 mg of the product obtained in Stage a) above (0.53 mmol), 1.06 ml of ethanol and 0.53 ml of NaOH (2N) (1.06 mmol, 2 eq.) are introduced, under N2, into a 10 cm3 flask. The medium is agitated at AT for 4 hours, diluted in 20 ml of H2O and then extracted twice with CH2Cl2/MeOH: 9/1 (2×25 ml). The organic phases are combined and washed with water saturated in NaCl, dried over MgSO4, filtered and evaporated to dryness. The expected product is obtained in the form of a yellow solid of 166 mg in weight.

Analyses:

¹H NMR spectrum: CDCl3 δ (ppm) 2.99, 1.57(dd): 2H; 2.06(bt), 3.0: 2H; 2.23(bd), 3.0: 2H; 2.32(s): CH3-N; 3.98(s), 4.01(s): 6H; 3.50(ddd): 1H(ax); 3.92(bs): 1H(eq); 3.26: 1H(OH); 6.48: 1H; 6.48: 1H; 8.57(dd): 1H; 7.44(dd): 1H; 7.92(dd): 1H. MS spectrum: MH+=431+; 413+IR spectrum: OH 3532 cm−1; C═O 1653 cm−1; conjugated system+aromatic: 1631, 1598, 1574, 1561, 1494 cm−1.

Stage c): 2-(2-chloro-3-pyridinyl)-5,7-dihydroxy-8-[(3S,4R)-3-hydroxy-1-methyl-4-piperidinyl]-4H-benzopyran-4-one trifluoroacetate

144 mg of the dimethoxy obtained in Stage b) (0.33 mmol), 3.3 cm3 of anhydrous DMF and 725 mg of Reillex-pyridine-HI (≈4 mmol/g) (2.9 mmol-10 eq.) obtained as indicated in Preparation 2) above are introduced, under N2, into a 30 cm3 flask with a reflux condenser. The reaction medium is taken to 130° C. for 42 hours, then returned to ambient temperature, diluted with CH3CN (10 ml), filtered on Iéna, rinsed with CH3CN (2×10 ml) then 2.6 g of PTBD resin (2.6 mmol/g) (6.7 mmol-20 eq.) is added. Agitation is carried out for 1 hour at ambient temperature, followed by filtering on Iéna, washing with CH3CN (100 ml) then releasing with 20 cm3 of CH3CN/TFA: 95/05 under agitation. After filtering on Iéna, rinsing with CH3CN/TFA: 95/05 (2×10 ml) then bringing to dryness under vacuum, 156 mg of an amorphous orange product is recovered which is taken up in 6 cm3 of HPLC eluent CH3CN/H2O/TFA: 25/75/0.1, filtered on a microporous filter-0.45 μm then injected 3 times into preparative HPLC: Kromasil C18—10 μm—500×22 mm—λ=225 nm—15 ml/min. The appropriate fractions are combined and concentrated under vacuum then lyophilized. In this way the expected product is isolated in the form of a yellow solid of 30 mg in weight.

Analyses:

¹H NMR spectrum: DMSO D⁶ δ (ppm) 1.82, 2.95: 2H; 2.73(bs): N—CH3; 3.07, 3.36: 2H; 3.29: 2H; 3.45(masked): 1H(ax); 4.07(bs): 1H(eq); 6.01: 1H(OH); 6.39(s): 1H; 6.71(s): 1H; 7.68(dd): 1H; 8.28(dd): 1H; 8.65(dd): 1H; 9.30, 11.34(s), 12.32(s): 3H(mobile). MS spectrum: MH+=403+

### EXAMPLE 14

### trifluoroacetate of 4-[8-[(3S,4R)-3-acetyloxy-1-methyl-4-piperidinyl]-5,7-dihydroxy-4-oxo-4H-benzopyran-2-yl]-2,5-dichloro-benzoic acid

Stage a): methyl 2,5-dichloro-4-[5,7-dimethoxy-8-[(3S,4R)-3-hydroxy-1-methyl-4-piperidinyl]-4-oxo-4H-benzopyran-2-yl]-benzoate

1) Condensation: methyl 2,5-dichloro-4-[3-[2-hydroxy-4,6-dimethoxy-3-[(3S,4R)-3-hydroxy-1-methyl-4-piperidinyl]phenyl]1,3-dioxopropyl]-benzoate 550 mg of tBuOK (M=112.22) (4.5 mmol-3 eq.) and 7 cm3 of DMF/NK20 are introduced, under N2, into a 30 cm3 flask. After cooling down to 0° C., 460 mg of Acetoflocinopiperidol (1.5 mmol) is added and agitation is carried out for 1 hour at ambient temperature, then 1079 mg of dimethyl 2,5-dichloro-1,4-benzenedicarboxylate (M=263.08) (4.5 mmol-3 eq.) is introduced. The reaction medium is maintained under agitation for 17 hours at ambient temperature, treated by pouring into water, the pH is adjusted to 7 by adding 1N HCl, followed by extracting 3 times with 20 cm3 of a CH2Cl2/MeOH: 90/10 mixture, washing with water and drying under vacuum. In this way 1.34 g of crude product is obtained.

2) Cyclization: methyl 2,5-dichloro-4-[5,7-dimethoxy-8-[(3S,4R)-3-hydroxy-1-methyl-4-piperidinyl]-4-oxo-4H -benzopyran-2-yl]-benzoate

1.3 g of the crude product obtained above in 1), 6.5 cm3 of DMF and 0.625 cm3 of 36% HCl (7.5 mmol-5 eq.) are placed in a flask under agitation. The reaction medium is then taken to 50° C. for 2 hours, returned to ambient temperature, poured into water and 0.65 cm3 of NaOH(12N) is added, followed by extracting 3 times with 20 cm3 of CH2Cl2/MeOH: 9/1, washing with water, drying over anhydrous MgSO4 and bringing to dryness under vacuum. 1.3 g of a brown oil is obtained which is chromatographed on silica 0.04-0.06 mm in CHCl3/MeOH: 90/10. The expected product is isolated in the form of a yellow solid of 210 mg in weight.

Analyses:

¹H NMR spectrum: CDCl3 δ (ppm) 1.56, 2.99: 2H; 3.99(s): 3H; 2.35(s): N—CH3; 3.97(s), 3.99(s): 6H; 2.03, 2.97: 2H; 2.20, 3.00: 2H; 3.91: 1H(eq); 3.45(ddd): 1H(ax); 3.19: 1H(OH); 6.45(s): 1H; 6.54(s): 1H; 7.30(s): 1H; 7.87(s): 1H.

Stage b): trifluoroacetate of 4-[8-[(3S,4R)-3-acetyloxy-1-methyl-4-piperidinyl]-5,7-dihydroxy-4-oxo-4H-benzopyran-2-yl]-2,5-dichloro-benzoic acid 130 mg of the dimethoxy obtained in Stage a) (0.25 mmol), 2.5 cm3 of anhydrous dimethylacetamide and 517 mg of pyridine-HI (M=207.1) (2.5 mmol-10 eq.) obtained as indicated above in Preparation 1) are introduced, under N2, into a 30 cm3 flask with a reflux condenser. The reaction medium is taken to 130° C. for 20 hours, then returned to ambient temperature, 2.5 cm3 of CH3CN is added, then 1.9 g of PTBD resin (2.6 mmol/g) (5 mmol-20 eq.) is added to the medium. Agitation is carried out for 2 hours at ambient temperature, followed by filtering on Iéna, washing with CH3CN then releasing with 10 cm3 of CH3CN/TFA: 95/05 under agitation, filtering on Iéna, rinsing with CH3CN/TFA: 95/05 then bringing to dryness under vacuum. 180 mg of an amorphous orange product is recovered which is taken up in 7 cm3 of HPLC eluent CH3CN/H2O/TFA: 27/73/0.1, filtered on a microporous filter-0.45 μm then injected 4 times into preparative HPLC: Kromasil C18—10 μm—500×22 mm—λ=225 nm.—15 ml/min. The appropriate fractions are combined and concentrated under vacuum then lyophilized. The expected product is isolated in the form of a a yellow solid of 32 mg in weight.

Analyses:

¹H NMR spectrum: DMSO D⁶ δ (ppm) 2(bd), 3.07(m): 2H; 3.16(m), 3.47(m): 2H; 3.50(m): 2H; 2.74(d): CH3-N⁺H; 5.11 (bs), 1.78 (s) : 1H(eq) ; 1.78(s): 3H; 3.59(masked): 1H(ax); 6.37(s): 1H; 6.68(s): 1H; 8.07(s), 8.03(s): 2H; 9.32(bs) : N⁺H; 11.39(s), 12.89(s), 14.0(bs) 3H(mobile).

### EXAMPLE 15

### hydrochloride of methyl 2,5-dichloro-4-[5,7-dihydroxy-8-[(3S,4R)-3-hydroxy-1-methyl-4-piperidinyl]-4-oxo-4H-benzopyran-2-yl]-benzoate

20 mg of the product of Example 14 (0.032 mmol), 0.5 cm3 of anhydrous MeOH and 0.01 cm3 of trimethylsilyl chloride (M=108.64 d=0.856) (2.5 eq 0.08 mmol) are introduced, under N2, into a 30 cm3 flask. The reaction medium is left for 24 hours at ambient temperature and another 0.05 cm3 of trimethylsilyl chloride is added. The reaction medium is taken to 50° C. for 20 hours, brought to dryness under vacuum with a rotary evaporator and the product is taken up in 20 cm3 of water and lyophilized. The expected product is obtained in the form of a yellow solid of 9.7 mg in weight.

### EXAMPLE 16

### 5,7-dihydroxy-2-(2,5-dimethyl-3-furanyl)-8-[(3S,4R)-3-hydroxy-1-methyl-4-piperidinyl]-4H-benzopyran-4-one trifluoroacetate

Stage a): 5,7-dimethoxy-2-(2,5-dimethyl-3-furanyl)-8-[(3S,4R)-3-hydroxy-1-methyl-4-piperidinyl]-4H-benzopyran-4-one

1) Condensation: 1-3-[2-hydroxy-4,6-dimethoxy-3-[(3S,4R)-3-hydroxy-1-methyl-4-piperidinyl]phenyl]-1,3-propanedione 310 mg of NaH at 50% in oil (M=24) (6.5 mmol-4 eq.), 10 cm3 of anhydrous THF, 500 mg of Acetoflocinopiperidol (1.6 mmol) by fractions, 2 drops of EtOH and 12 mg of Dibenzo-18-Crown-6 (M=360.41) (0.03 mmol-2% molar) are introduced, under N2, into a 30 cm3 flask. Agitation is carried out for 5 minutes at ambient temperature, then 685 μl of methyl 2,5-dimethyl-3-furancarboxylate (M=154.17, d=1.092) (4.8 mmol-3 eq.) is introduced. The reaction medium is agitated for 17 hours at ambient temperature, then taken to reflux for 4 hours 30 minutes.

2) Cyclization: 5,7-dimethoxy-2-(2,5-dimethyl-3-furanyl)-8-[(3S,4R)-3-hydroxy-1-methyl-4-piperidinyl]-4H-benzopyran-4-one

10 cm3 of 36% HCl (116 mmol-72 eq.) is added to the reaction medium obtained above in 1), and maintained under reflux for 2 hours. After returning to ambient temperature, the reaction medium is poured into 100 cm3 of NaOH(2N), followed by extracting with 2×100 cm3 of CH2Cl2/MeOH: 8/2, washing with a saturated solution of NaCl, drying over anhydrous Na2SO4 and bringing to dryness under vacuum. 2.13 g of a brown oil is obtained which is chromatographed successively on silica 0.04-0.06 mm in CH2Cl2/MeOH: 80/20 then in CH2Cl2/MeOH: 90/10. The expected product is isolated in the form of a brown resin of 89 mg in weight.

Analyses:

¹H NMR spectrum: DMSO D⁶ δ (ppm) 1.46(d), 3.05(m): 2H; 1.98(t), 2.90(m): 2H; 2.17(d), 2.80(m): 2H; 2.22(s), 2.28(s): 6H; 2.52(s): N—CH3; 3.32,(masked): 1H; 3.73(bs): 1H(eq); 3.87(s), 3.91(s): 6H; 6.14(s): 1H; 6.49(s): 1H; 6.61(s): 1H.

Stage b): 5,7-dihydroxy-2-(2,5-dimethyl-3-furanyl)-8-[(3S,4R)-3-hydroxy-1-methyl-4-piperidinyl]-4H-benzopyran-4-one trifluoroacetate

89 mg of the dimethoxy obtained above in Stage a) (0.22 mmol), 2.5 cm3 of anhydrous DMF and 538 mg of Reillex-pyridine-HI (≈4 mmol/g) (2.15 mmol-10 eq.) obtained in Preparation 2) above are introduced, under N2, into a 30 cm3 flask with a reflux condenser. The reaction medium is taken to 130° C. for 20 hours 30 minutes then at 150° C. for 3 hours, then returned to ambient temperature, diluted with CH3CN, filtered on Iéna, rinsed with CH3CN then 1.7 g of PTBD resin (2.6 mmol/g) (4.4 mmol-20 eq.) is added. Agitation is caried out for 2 hours 30 minutes at ambient temperature, followed by filtering on Iéna, washing with CH3CN then releasing with 20 cm3 of CH3CN/TFA: 95/05 under agitation, filtering on Iéna, rinsing with CH3CN/TFA: 95/05 then bringing to dryness under vacuum. 135 mg of a brown oil is recovered which is taken up in 3 cm3 of water and a few drops of CH3CN then deposited on a 2 g cartridge of C18. The cartridge is eluted successively with 10 cm3 of CH3CN/H2O: 5/95, 20 cm3 of CH3CN/H2O: 20/80, 40 cm3 of CH3CN/H2O: 50/50 and 20 cm3 of pure CH3CN. The appropriate fractions are combined and concentrated under vacuum then lyophilized. The expected product is isolated in the form of a pale yellow solid of 49 mg in weight.

Analyses:

¹H NMR spectrum: DMSO D⁶ δ (ppm) 1.80(d), 3.11(masked): 2H; 3.11, 3.47(masked): 2H; 2.32(s), 2.59(s): 6H; 2.81(s): N—CH3; 3.27(d), 3.41(masked): 2H; 3.52(masked): 1H(ax); 4.15(bs): 1H(eq); 6.31(s): 1H; 6.42(s): 1H; 6.62(s): 1H; 9.30(bs), 11.17(bs), 13.22(s): 3H(mobile).

### EXAMPLE 17

### 2-[4-(diethylamino)-phenyl]-5,7-dihydroxy-8-[(3S,4R)-3-hydroxy-1-methyl-4-piperidinyl]-4H-benzopyran-4-one trifluoroacetate

Stage a): 2-[4-(diethylamino)-pheny]-5,7-dimethoxy-8-[(3S,4R)-3-hydroxy-1-methyl-4-piperidinyl]-4H-benzopyran-4-one

1) Condensation: 1-[4-(diethylamino)-pheny]-3-[2-hydroxy-4,6-dimethoxy-3-[(3S,4R)-3-hydroxy-1-methyl-4-piperidinyl]phenyl]-1,3-propanedione

310 mg of NaH at 50% in oil (M=24) (6.5 mmol-4 eq.), 10 cm3 of anhydrous THF, 500 mg of Acetoflocinopiperidol (1.6 mmol) by fractions, 2 drops of EtOH and 12 mg of Dibenzo-18-Crown-6 (M=360.41) (0.03 mmol-2% molar) are introduced, under Ar, into a dry 30 cm3 tube. Agitation is carried out for 1 hour at ambient temperature, then 1.00 g of methyl 4-(diethylamino)-benzoate (M=207.27) (4.8 mmol-3 eq.) is introduced. The reaction medium is maintained under agitation at ambient temperature, for 19 hours then taken to reflux for 22 hours 30 minutes, then returned to ambient temperature.

2) Cyclization: 2-[4-(diethylamino)-pheny]-5,7-dimethoxy-8-[(3S,4R)-3-hydroxy-1-methyl-4-piperidinyl]-4H-benzopyran-4-one

10 cm3 of 36% HCl (116 mmol-72 eq) is added to the reaction medium obtained above in 1) and the whole is then taken to 60° C. for 2 hours. After returning to ambient temperature, the reaction medium is poured into 100 cm3 of NaOH (2N), followed by extracting with 2×200 cm3 of CH2Cl2/MeOH: 8/2, washing with a saturated solution of NaCl, drying over anhydrous Na2SO4 and bringing to dryness under vacuum. 1.28 g of a brown oil is obtained which is chromatographed on silica 0.04-0.06 mm in CH2Cl2/MeOH: 90/10. The expected amorphous yellow product is isolated weighing 152 mg.

Analyses:

IR spectrum: —OH complex: 3520 cm-1; —C═O: 1655 cm-1; conjugated system+aromatic: 1621, 1596, 1568, 1494 cm-1. ¹H NMR spectrum: CDCl3 δ (ppm) 1.23(t): 6H; 3.45(q): 4H; 1.61(bd), 3.12(masked): 2H; 2.16(bt), 3.09(masked): 2H; 2.33(bd), 3.15(masked): 2H; 3.64(ddd): 1H(axial); 2.41(s): CH3-N; 3.96(s), 3.99(s): 6H; 2.81(s): N—CH3; 4.04(bs): 1H(eq); 6.43(s): 1H; 6.53(s): 1H; 6.73, 7.66 AA′BB′: 4H.

Stage b): 2-[4-(diethylamino)-phenyl]-5,7-dihydroxy-8-[(3S,4R)-3-hydroxy-1-methyl-4-piperidinyl]-4H-benzopyran-4-one trifluoroacetate

140 mg of the dimethoxy obtained in Stage a) above (0.30 mmol), 3 cm3 of anhydrous DMF and 750 mg of Reillex-pyridine-HI (≈4 mmol/g) (3.0 mmol-10 eq.) obtained as indicated in Preparation 2) above are introduced, under N2, into a 30 cm3 flask with a reflux condenser. The reaction medium is taken to 130° C. for 37 hours and to 150° C. for 8 hours, then returned to ambient temperature, diluted with CH3CN, filtered on Iéna, rinsed with CH3CN then 2.3 g of PTBD resin (2.6 mmol/g) (6 mmol-20 eq.) is added. Agitation is carried out for 1 hour at ambient temperature, followed by filtering on Iéna, washing with CH3CN then releasing with 20 cm3 of CH3CN/TFA: 95/05 under agitation, filtering on Iéna, rinsing with CH3CN/TFA: 95/05 then bringing to dryness under vacuum. 304 mg of an amorphous orange product is recovered which is taken up in 4 cm3 of HPLC eluent: CH3CN/H20/TFA: 30/70/0.1, filtered on a microporous filter-0.45 μm then injected 4 times into preparative HPLC Kromasil C18—10 μm—500×22 mm—λ=225 nm—15 ml/min. The appropriate fractions are combined and concentrated under vacuum then lyophilized. The expected product is isolated in the form of a yellow solid of 109 mg in weight.

Analyses:

¹H NMR spectrum: DMSO D⁶δ (ppm) 1.15(t): 6H; 3.45(masked): 4H; 1.81(d), 3.21(masked): 2H; 3.21, 3.43(masked): 2H; 3.42(m): 2H; 3.52(masked): 1H (ax); 4.10(s): 1H (eq); 2.80(s): N—CH3; 6.29(s): 1H; 6.69(s): 1H; 6.80, 7.91 AA′BB′: 4H; 6.02(bs), 9.41(bs), 10.48(bs), 13.47(bs): 4H (mobile).

### EXAMPLE 18

### 5,7-dihydroxy-8-[(3S,4R)-3-hydroxy-1-methyl-4-piperidinyl]-2-[3-(trifluoromethoxy)-phenyl]-4H-benzopyran-4-one trifluoroacetate

Stage a): 5,7-dimethoxy-8-[(3S,4R)-3-hydroxy-1-methyl-4-piperidinyl]-2-[3-(trifluoromethoxy)-phenyl]-4H-benzopyran-4-one

1) Condensation: 1-[2-hydroxy-4,6-dimethoxy-3-[(3S,4R)-3-hydroxy-1-methyl-4-piperidinyl]phenyl]-3-[3-(trifluoro-methoxy)-phenyl]-1,3-propanedione

310 mg of NaH at 50% in oil (M=24) (6.5 mmol-4 eq.), 10 cm3 of anhydrous THF, 500 mg of Acetoflocinopiperidol (1.6 mmol) by fractions, 2 drops of EtOH and 12 mg of Dibenzo-18-Crown-6 (M=360.41) (0.03 mmol-2% molar) are introduced, under N2, into a dry 50 cm3 flask. Agitation is carried out for 30 minutes at ambient temperature, then 1.1 g of methyl 3-(trifluoromethoxy)-benzoate (M=220.15) (5.0 mmol-3 eq.) is introduced. The reaction medium is maintained under agitation at ambient temperature for 18 hours.

2) Cyclization: 5,7-dimethoxy-8-[(3S,4R)-3-hydroxy-1-methyl-4-piperidinyl]-2-[3-(trifluoromethoxy)-phenyl]-4H-benzopyran-4-one

10 cm3 of 36% HCl (116 mmol-72 eq.) is added to the reaction medium obtained above in 1). The reaction medium is then taken to 55° C. for 2 hours, returned to ambient temperature, poured into 100 cm3 of NaOH(2N), extracted with 2×200 cm3 of CH2Cl2/MeOH: 8/2, washed with a saturated solution of NaCl, dried over anhydrous Na2SO4 and brought to dryness under vacuum. 1.33 g of an orange oil is obtained which is chromatographed on silica 0.04-0.06 mm in CH2Cl2/MeOH/NH40H: 90/10/1. The expected product is isolated in the form of cream solid of 384 mg in weight.

Analyses:

IR spectrum:—Absorption OH/NH region —C═O: 1645 cm-1; —C═C+aromatic: 1622, 1596, 1568 cm-1. ¹H NMR spectrum: DMSO D⁶ δ (ppm) 1.46(bd), 3.07(m): 2H; 1.93(bt), 2.88(masked): 2H; 2.16(masked), 2.90(masked): 2H; 2.20(s): CH3-N; 3.33 (masked): 1H(ax); 3.77(bs): 1H(eq); 3.89(s), 3.93(s): 6H; 4.32(bd): 1H(OH); 6.65(s): 1H; 6.82 (s): 1H; 7.59(bd): 1H; 8.15(bd): 1H; 7.71(t): 1H; 8.13(bs): 1H.

Stage b): 5,7-dihydroxy-8-[(3S,4R)-3-hydroxy-1-methyl-4-piperidinyl]-2-[3-(trifluoromethoxy)-phenyl]-4H-benzopyran-4-one trifluoroacetate

106 mg of the dimethoxy obtained in Stage a) above (0.22 mmol), 2.5 cm3 of anhydrous DMF and 538 mg of Reillex-pyridine-HI (≈4 mmol/g) (2.15 mmol-10 eq.) obtained as indicated in Preparation 2) above are introduced, under N2, into a 30 cm3 flask with a reflux condenser. The reaction medium is taken to 130° C. for 18 hours and to 150° C. for 7 hours, then returned to ambient temperature, diluted with CH3CN, filtered on Iéna, rinsed with CH3CN then 1.7 g of PTBD resin (2.6 mmol/g) (4.4 mmol-20 eq.) is added. Agitation is carried out for 2 hours at ambient temperature, followed by filtering on Iéna, washing with CH3CN then releasing with 20 cm3 of CH3CN/TFA: 95/05 under agitation, filtering on Iéna, rinsing with CH3CN/TFA: 95/05 then bringing to dryness under vacuum. 152 mg of a yellow resin is recovered which is taken up in 2 cm3 of HPLC eluent: CH3CN/H20/TFA: 40/60/0.1, filtered on a microporous filter-0.45 μm then injected twice into preparative HPLC: Kromasil C18—10 μm—500×22 mm λ=225 nm.—15 ml/min. The appropriate fractions are combined and concentrated under vacuum then lyophilized. The expected product is isolated in the form of a yellow solid of 88 mg in weight.

Analyses:

¹H NMR spectrum: DMSO D⁶ δ (ppm) 1.87(bd), 3.15(m): 2H; 2.79(bs): CH3-N; 3.19(m), 3.45(m): 2H; 3.38(m): 2H; 3.55(masked): 1H(ax); 4.11(bs): 1H(eq); 6.37(s): 1H; 7.05(s): 1H; 5.99(bs): 1H (OH); 7.67(bd): 1H; 7.77(bt): 1H; 8.05(bs): 1H; 8.17(bd): 1H; 9.43(bs), 11.26(bs), 13.08(s): 3H (mobile).

### EXAMPLE 19

### 2-[3,5-bis(trifluoromethyl)-pheny]-5,7-dihydroxy-8-[(3S,4R)-3-hydroxy-1-methyl-4-piperidinyl]-4H-benzopyran-4-one trifluoroacetate

Stage a): 2-[3,5-bis(trifluoromethyl)-phenyl]-5,7-dimethoxy-8-[(3S,4R)-3-hydroxy-1-methyl-4-piperidinyl]-4H-benzopyran-4-one

1) Condensation: 1-[3,5-bis(trifluoromethyl)-pheny]-3-[2-hydroxy-4,6-dimethoxy-3-[(3S,4R)-3-hydroxy-1-methyl-4-piperidinyl]phenyl]-1,3-propanedione

310 mg of NaH at 50% in oil (M=24) (6.5 mmol-4 eq.), 10 cm3 of anhydrous THF, 500 mg of Acetoflocinopiperidol (1.6 mmol) by fractions, 2 drops of EtOH and 12 mg of Dibenzo-18-Crown-6 (M=360.41) (0.03 mmol-2% molar), are introduced, under N2, into a dry 50 cm3 flask. Agitation is carried out for 30 minutes at ambient temperature, then 1.3 g of methyl 3,5-bis(trifluoromethyl)-benzoate (M=272.15) (4.8 mmol-3 eq.) is introduced and the reaction medium is maintained under agitation at ambient temperature for 18 hours.

2) Cyclization: 2-[3,5-bis(trifluoromethyl)-phenyl]-5,7-dimethoxy-8-[(3S,4R)-3-hydroxy-1-methyl-4-piperidinyl]-4H-benzopyran-4-one

10 cm3 of 36% HCl (116 mmol-72eq.) is added to the reaction medium obtained above in 1) and then the medium is taken to 55° C. for 2 hours, returned to ambient temperature, poured into 100 cm3 of NaOH (2N), extracted with 2×200 cm3 of CH2Cl2/MeOH: 8/2, washed with a saturated solution of NaCl, dried over anhydrous Na2SO4 and brought to dryness under vacuum. 985 mg of an orange solid is obtained which is impasted in 15 cm3 of isopropyl ether under reflux, then left to return to ambient temperature, filtered then dried under vacuum. The expected product is isolated in the form of a yellow solid of 503 mg in weight.

Analyses:

IR spectrum:—Absorption OH/NH region —C═O: 1660 cm-1; —C═C+aromatic: 1632, 1622, 1573, 1565 cm-1.

Analyses:

¹H NMR spectrum: DMSO D⁶ δ (ppm) 1.49(bd), 3.05(ddt): 2H; 1.93(bt), 2.89(bs): 2H; 2.16 (masked), 2.88(bs): 2H; 2.20(s): CH3-N; 3.35(masked): 1H (ax); 3.78(bs): 1H(eq); 3.90(s), 3.93(s): 6H; 4.29(bd): 1H (OH); 6.66(s): 1H; 7.15(s): 1H; 8.31(bs), 8.76(bs): 3H.

Stage b): 2-[3,5-bis(trifluoromethyl)-pheny]-5,7-dihydroxy-8-[(3S,4R)-3-hydroxy-1-methyl-4-piperidinyl]-4H-benzopyran-4-one trifluoroacetate

117 mg of the dimethoxy obtained in Stage a) above (0.22 mmol), 2.5 cm3 of anhydrous DMF and 538 mg of Reillex-pyridine-HI (≈4 mmol/g) (2.15 mmol-10 eq.) obtained as indicated in Preparation 2) above are introduced, under N2, into a 20 cm3 flask with a reflux condenser. The reaction medium is taken to 150° C. for 6 hours and to 130° C. for 19 hours, then returned to ambient temperature, diluted with CH3CN, filtered on Iéna, rinsed with CH3CN then 1.7 g of PTBD resin (2.6 mmol/g) (4.4 mmol-20 eq.) is added. Agitation is carried out for 2 hours at ambient temperature, followed by filtering on Iéna, washing with CH3CN then releasing with 20 cm3 of CH3CN/TFA: 95/05 under agitation, filtering on Iéna, rinsing with CH3CN/TFA: 95/05 then bringing to dryness under vacuum. 158 mg of a yellow resin is recovered which is taken up in 2 cm3 of HPLC eluent: CH3CN/H20/TFA: 45/55/0.1, filtered on a microporous filter-0.45 μm then injected twice into preparative HPLC: Kromasil C18—10 μm—500×22 mm—λ=225 nm—15 ml/min. The appropriate fractions are combined and concentrated under vacuum then lyophilized. The expected product is isolated in the form of a slightly yellow solid of 41 mg in weight.

Analyses:

¹H NMR spectrum: DMSO D⁶ δ (ppm) 2.05(bd), 3.15(m): 2H; 2.76(bs): CH3-N; 3.14(m), 3.40(masked): 2H; 3.21(m), 3.40(masked): 2H; 3.59(bd): 1H(ax); 4.18(bs): 1H(eq); 6.38(s): 1H; 7.30 (s): 1H; 8.43(s): 1H; 8.62(s): 1H; 5.96(bs), 9.46(bs), 11.25(bs), 12.93(bs): 4H(mobile).

### EXAMPLE 20

### 2-(2,6-dichloro-4-pyridinyl)-5,7-dihydroxy-8-[(3S,4R)-3-hydroxy-1-methyl-4-piperidinyl]-4H-benzopyran-4-one trifluoroacetate

Stage a): 2-(2,6-dichloro-4-pyridinyl)-5,7-dimethoxy-8-[(3S,4R)-3-hydroxy-1-methyl-4-piperidinyl]-4H-benzopyran-4-one

1) Condensation: 1-(2,6-dichloro-4-pyridinyl)-3-[2-hydroxy-4,6-dimethoxy-3-[(3S,4R)-3-hydroxy-1-methyl-4-piperidinyl]phenyl]-1,3-propanedione

310 mg of NaH at 50% in oil (M=24) (6.5 mmol-4 eq.), 10 cm3 of anhydrous THF, 500 mg of Acetoflocinopiperidol (1.6 mmol) by fractions, 2 drops of EtOH and 12 mg of Dibenzo-18-Crown-6 (M=360.41) (0.03 mmol-2% molar) are introduced, under N2, into a 30 cm3 flask. Agitation is carried out for 30 minutes at ambient temperature, then 1.00 g of methyl 2,6-dichloro-4-pyridinecarboxylate (M=360.41) (4.8 mmol-3 eq.) is introduced. The reaction medium is maintained under agitation at ambient temperature for 18 hours.

2) Cyclization: 2-(2,6-dichloro-4-pyridinyl)-5,7-dimethoxy-8-[(3S,4R)-3-hydroxy-1-methyl-4-piperidinyl]-4H-benzopyran-4-one

10 cm3 of 36% HCl (116 mmol-72 eq.) is added to the reaction medium obtained above in 1), and the medium is then taken to 50° C. for 2 hours, returned to ambient temperature, poured into 100 cm3 of NaOH(2N), extracted with 2×200 cm3 of CH2Cl2/MeOH: 8/2, washed with a saturated solution of NaCl, dried over anhydrous Na2SO4 and brought to dryness under vacuum. 820 mg of a yellow solid is obtained which is chromatographed on silica 0.04-0.06 mm in CH2Cl2/MeOH/NH40H: 90/10/1. The expected product is isolated in the form of a pale yellow solid of 384 mg in weight.

Analyses:

IR spectrum:—Absorption OH/NH region —C═O: 1658 cm-1; conjugated system+aromatic: 1632, 1595, 1585, 1568, 1528 cm-1. ¹H NMR spectrum: DMSO D⁶ δ (ppm) 1.53(bd), 3.06(masked): 2H; 2.09(bt), 2.97(masked): 2H; 2.31(masked), 2.97(masked): 2H; 3.83(masked): 1H (eq); 3.37(bd): 1H; 2.29(bs): CH3-N; 3.90(s), 3.93(s): 6H; 6.66(s): 1H; 6.98(s): 1H; 8.16(s): 2H.

Stage b): 2-(2,6-dichloro-4-pyridinyl)-5,7-dihydroxy-8-[(3S,4R)-3-hydroxy-1-methyl-4-piperidinyl]-4H-benzopyran-4-one trifluoroacetate

150 mg of the dimethoxy obtained in Stage a) above (0.32 mmol), 3.5 cm3 of anhydrous DMF and 806 mg of Reillex-pyridine-HI (≈4 mmol/g) (3.22 mmol-10 eq.) obtained as indicated in Preparation 2) above are introduced, under N2, into a 50 cm3 flask with a reflux condenser. The reaction medium is taken to 130° C. for 33 hours and to 150° C. for 12 hours 30 minutes, then returned to ambient temperature, diluted with CH3CN, filtered on Iéna, rinsed with CH3CN then 2.5 g of PTBD resin (2.6 mmol/g) (6.5 mmol-20 eq.) is added. Agitation is carried out for 1 hour 30 minutes at ambient temperature, followed by filtering on Iéna, washing with CH3CN then releasing with 40 cm3 of CH3CN/TFA: 95/05 under agitation, filtering on Iéna, rinsing with CH3CN/TFA: 95/05 then bringing to dryness under vacuum. 211 mg of a yellow resin is recovered which is taken up in 5 cm3 of HPLC eluent: CH3CN/H2O/TFA: 33/67/0.1, filtered on a microporous filter-0.45 μm then injected 5 times into preparative HPLC: Kromasil C18—10 μm—500×22 mm λ=225 nm—15 ml/min. The appropriate fractions are combined and concentrated under vacuum then lyophilized. The expected product is isolated in the form of a yellow solid of 39 mg in weight.

Analyses:

¹H NMR spectrum: DMSO D⁶ δ (ppm) 1.91(bd), 3.11(m): 2H; 3.20(m), 3.44(masked): 2H; 3.31(m), 3.39(masked): 2H; 2.79(bs): CH3-N; 3.56(bd): 1H(ax); 4.15(bs): 1H(eq); 6.38(s): 1H; 7.25(s): 1H; 8.11(bs): 2H; 9.37(bs), 11.39(bs), 12.82(s), 6.04(bs): 4H (mobile).

### EXAMPLE 21

### 2-[5-bromo-2-furanyl]-5,7-dihydroxy-8-[(3S,4R)-3-hydroxy-1-methyl-4-piperidinyl]-4H-benzopyran-4-one trifluoroacetate

Stage a): 2-[5-bromo-2-furanyl]-5,7-dimethoxy-8-[(3S,4R)-3-hydroxy-1-methyl-4-piperidinyl]-4H-benzopyran-4-one

1) Condensation: 1-[5-bromo-2-furanyl]-3-[2-hydroxy-4,6-dimethoxy-3-[(3S,4R)-3-hydroxy-1-methyl-4-piperidinyl]phenyl]-1,3-propanedione

310 mg of NaH at 50% in oil (M=24) (6.5 mmol-4 eq.), 10 cm3 of anhydrous THF, 500 mg of Acetoflocinopiperidol (1.6 mmol) by fractions, 2 drops of EtOH and 12 mg of Dibenzo-18-Crown-6 (M=360.41) (0.03 mmol-2% molar) are introduced, under N2, into a 30 cm3 flask. Agitation is carried out for 10 minutes at ambient temperature, then 994 mg of methyl 5-bromo-2-furancarboxylate (M=201.05) (4.85 mmol-3 eq.) is introduced. The reaction medium is maintained under agitation at ambient temperature for 18 hours.

2) Cyclization: 2-[5-bromo-2-furanyl]-5,7-dimethoxy-8-[(3S,4R)-3-hydroxy-1-methyl-4-piperidinyl]-4H-benzopyran-4-one

10 cm3 of 36% HCl (116 mmol-72 eq.) is added to the reaction medium obtained above in 1) and then the medium is taken to 50° C. for 1 hour 30 minutes, returned to ambient temperature, poured into 100 cm3 of NaOH (2N), extracted with 2×100 cm3 of CH2Cl2/MeOH: 8/2, washed with a saturated solution of NaCl, dried over anhydrous Na2SO4 and brought to dryness under vacuum. 1.48 g of a brown oil is obtained which is chromatographed on silica 0.04-0.06 mm in CH2Cl2/MeOH: 90/10. The expected amorphous yellow product is isolated of 321 mg in weight.

Analyses:

IR spectrum: Absorption OH/NH region —C═O: 1655 cm-1; conjugated system+aromatic: 1621, 1596, 1568, 1494 cm-1. ¹H NMR spectrum: DMSO D⁶ δ (ppm) 1.53(bd), 3.10(bd): 2H; 2.37(masked), 2.97(bd): 2H; 2.15(bt), 3.01(bd): 2H; 2.30(s): CH3-N; 3.30(masked): 1H(ax); 3.76(bs): 1H(eq); 3.88(s), 3.91(s): 6H; 4.36(bs): 1H (OH); 6.33(s): 1H; 6.63(s): 1H; 6.95(d), 7.37(d): 2H.

Stage b): 2-[5-bromo-2-furanyl]-5,7-dihydroxy-8-[(3S,4R)-3-hydroxy-1-methyl-4-piperidinyl]-4H-benzopyran-4-one trifluoroacetate

140 mg of the dimethoxy obtained in Stage a) above (0.3 mmol), 3 cm3 of anhydrous DMF and 754 mg of Reillex-pyridine-HI (≈4 mmol/g) (3 mmol-10 eq.) obtained as indicated in Preparation 2) above are introduced, under N2, into a 30 cm3 flask with a reflux condenser. The reaction medium is taken to 130° C. for 40 hours 30 minutes and to 150° C. for 2 hours, then returned to ambient temperature, diluted with CH3CN, filtered on Iéna, rinsed with CH3CN then 2.3 g of PTBD resin (2.6 mmol/g) (6.0 mmol-20 eq.) is added. Agitation is carried out for 1 hour 30 minutes at ambient temperature, followed by filtering on Iéna, washing with CH3CN then releasing with 10 cm3 of CH3CN/TFA: 95/05 under agitation, filtering on Iéna, rinsing with CH3CN/TFA: 95/05 then bringing to dryness under vacuum. 287 mg of an amorphous brown product is recovered which is taken up in 4 cm3 of HPLC eluent: CH3CN/H20/TFA: 30/70/0.1, filtered on a microporous filter-0.45 μm then injected twice into preparative HPLC: Kromasil C18—10 μm—500×22 mm—λ=225 nm—15 ml/min. The appropriate fractions are combined and concentrated under vacuum then lyophilized. The expected product is isolated in the form of a yellow solid of 44 mg in weight.

Analyses:

¹H NMR spectrum: DMSO D⁶ δ (ppm) 1.80(m), 3.17(masked): 2H; 2.81(d): NH⁺—CH3; 3.18(m), 3.47(m): 2H; 3.39(m): 2H; 3.48(masked): 1H(ax); 4.04(s): 1H(eq); 6.34(s), 6.52(s): 2H; 7.03(d), 7.56(d): 2H; 5.79(s), 9.39(s), 11.24(s), 13.05(s): 4H(mobile).

### EXAMPLE 22

### 8-[(3S,4R)-3-acetyloxy-1-methyl-4-piperidinyl]-2-(5-bromo-2-furanyl) 5,7-dihydroxy-4H-benzopyran-4-one trifluoroacetate

140 mg of the product obtained in Stage a) of Example 21 (0.30 mmol), 3 cm3 of DMA and 625 mg of Pyridine-HI (M =207.01) (3.0 mmol-10 eq.) obtained as indicated in Preparation 1) above are introduced, under N2, into a flask with a reflux condenser. The reaction medium is taken to 130° C. for 40 hours and to 150° C. for 5 hours 30 minutes, then returned to ambient temperature, diluted with CH3CN, then 2.3 g of PTBD resin (2.6 mmol/g) (6.0 mmol-20 eq.) is added. The reaction medium is agitated for 1 hour 20 minutes at ambient temperature, followed by filtering on Iéna, washing with CH3CN then releasing with 50 cm3 of CH3CN/TFA: 95/05 then bringing to dryness under vacuum. 241 mg of a red resin is recovered which is taken up in 8 cm3 of HPLC eluent CH3CN/H2O/TFA: 35/65/0.1, described hereafter, filtered on a microporous filter-0.45 μm then injected 4 times into preparative HPLC: Kromasil C18—10 μm—500×22 mm—λ=225 nm—15 ml/min. The appropriate fractions are combined and brought to dryness under vacuum, taken up in water then lyophilized. The expected product is isolated in the form of a cream solid of 47 mg in weight.

Analyses:

¹H NMR spectrum: DMSO D⁶ δ (ppm) 1.94(bd), 3.21(m): 2H; 2.85(s): CH3-N; 3.26(masked), 3.59(m): 2H; 3.59(m): 2H; 3.68(bt): 1H(ax); 5.09(bs): 1H(eq); 6.33(s), 6.52(s): 2H; 7.03(d), 7.56(d): 2H; 9.82(bs) : N⁺H; 11.38(s), 13.06(s): 2H; 1.78(s): 3H.

### EXAMPLE 23

### 2-cyclohexyl-5,7-dihydroxy-8-[(3S,4R)-3-hydroxy -1-methyl-4-piperidinyl]-4H-benzopyran-4-one trifluoroacetate

Stage a): 2-cyclohexyl-5,7-dimethoxy-8-[(3S,4R)-3-hydroxy-1-methyl-4-piperidinyl]-4H-benzopyran-4-one

1) Condensation: 1-cyclohexyl-3-[2-hydroxy-4,6-dimethoxy-3-[(3S,4R)-3-hydroxy-1-methyl-4-piperidinyl]phenyl]-1,3-propanedione

370 mg of NaH at 50% in oil (M=24) (7.7 mmol-4 eq.), 20 cm3 of DMSO/NK20 and 600 mg of Acetoflocinopiperidol (1.9 mmol) are introduced, under N2, into a dry 50 cm3 flask. Agitation is carried out for 1 hour at ambient temperature, then 0.85 cm3 of methyl cyclohexanecarboxylate (M=142.2-d=0.995) (5.95 mmol-3 eq.) is introduced and the reaction medium is maintained under agitation for 22 hours.

2) Cyclization: 2-cyclohexyl-5,7-dimethoxy-8-[(3S,4R)-3-hydroxy-1-methyl-4-piperidinyl]-4H-benzopyran-4-one 13 cm3 of 36% HCl (151 mmol-78 eq.) is added to the reaction medium obtained above in 1). The reaction medium is then taken to 50° C. for 2 hours 15 minutes, returned to ambient temperature, poured into 200 cm3 of NaOH(N), extracted with 200 cm3 of CH2Cl2 then with 200 cm3 of CH2Cl2/MeOH: 9/1, washed with a saturated solution of NaCl, dried over anhydrous Na2SO4 and brought to dryness under vacuum. 1.173 g of a brown oil is obtained which is chromatographed on silica 0.04-0.06 mm in CH2Cl2/MeOH: 90/10. The expected product is isolated in the form of a yellow solid of 511 mg in weight.

Analyses:

IR spectrum: —OH: 3535 cm−1; —C═O: 1652 cm−1; —C═C+aromatic: 1619, 1597, 1495 cm−1. ¹H NMR spectrum: CDCl3 δ (ppm) 1.25(m), 1.77(m): 2H; 1.86(m), 1.42(m): 4H; 1.41(m), 2.50(m): 4H; 2.49(tt): 1H; 1.62(m), 3.13(m): 2H; 2.17(m), 3.11(m): 2H; 2.32(d), 3.13(m): 2H; 2.41(s): N—CH3; 3.94(s), 3.96(s): 6H; 3.43(ddd): 1H (ax); 3.90(masked): 1H (eq); 6.00(s): 1H; 6.39(s): 1H.

Stage b): 2-cyclohexyl-5,7-dihydroxy-8-[(3S,4R)-3-hydroxy-1-methyl-4-piperidinyl]-4H-benzopyran-4-one trifluoroacetate 100 mg of the dimethoxy obtained in Stage a) above (0.25 mmol) and 1.3 cm3 of 57% HI are introduced, under N2, into a 20 cm3 flask with a reflux condenser. The reaction medium is taken to 130° C. for 1 hour, cooled down to 0° C. with an ice bath then rendered basic with 2 cm3 of NaOH (≈6 M) in MeOH, injected twice into preparative HPLC: Kromasil C18—10 μm—500×22 mm λ=225 nm—15 ml/min. with MeOH/H2O/TFA: 55/45/0.1 as eluent. The appropriate fractions are combined and brought to dryness under vacuum, taken up in 1 cm3 of MeOH+100 cm3 of water then lyophilized. In this way the expected product is isolated in the form of a white solid of 29.4 mg in weight.

Analyses:

¹H NMR spectrum: DMSO D⁶ δ (ppm) 1.75, 1.42: 2H; 1.36, 1.85: 4H; 1.95, 1.52: 4H; 2.61: 1H; 2.82: CH3-N; 3.39: 2H; 4.05: 1H; 3.40: 1H; 1.83, 3.48: 2H; 5.80: 1H(OH); 6.16(s): 1H; 6.33(s): 1H; 13.15, 11.14: 2H(mobile).

### EXAMPLE 24

### 5,7-dihydroxy-8-[(3S,4R)-3-hydroxy-1-methyl-4-piperidinyl]-2-[4-(trifluoromethoxy)-phenyl]-4H-benzopyran-4-one trifluoroacetate

Stage a): 5,7-dimethoxy-8-[(3S,4R)-3-hydroxy-1-methyl-4-piperidinyl]-2-[4-(trifluoromethoxy)-phenyl]-4H-benzopyran-4-one

1) Condensation: 1-[2-hydroxy-4,6-dimethoxy-3-[(3S,4R)-3-hydroxy-1-methyl-4-piperidinyl]phenyl]-3-[4-(trifluoro-methoxy)-phenyl]-1,3-propanedione

310 mg of NaH at 50% in oil (M=24) (6.5 mmol-4 eq.), 16 cm3 of DMSO/NK20 and 500 mg of Acetoflocinopiperidol (1.6 mmol) by fractions are introduced, under N2, into a dry 50 cm3 flask. Agitation is carried out for 1 hour at ambient temperature, then 1.07 g of methyl 4-(trifluoromethoxy)-benzoate (M=220.16) (4.9 mmol-3 eq.) is introduced. The reaction medium is maintained under agitation for 17 hours at ambient temperature, then heated to 50° C. for 2 hours.

2) Cyclization: 5,7-dimethoxy-8-[(3S,4R)-3-hydroxy-1-methyl-4-piperidinyl]-2-[4-(trifluoromethoxy)-phenyl]-4H-benzopyran-4-one 11 cm3 of 36% HCl (128 mmol-79 eq.) is added to the reaction medium obtained above in 1), and the medium is maintained at 50° C. for 1 hour 30 minutes, returned to ambient temperature, poured into 70 cm3 of NaOH(2N), extracted with 2×100 cm3 of CH2Cl2/MeOH: 8/2, washed with a saturated solution of NaCl, dried over anhydrous Na2SO4 and brought to dryness under vacuum. 1.23 g of a yellow oil is obtained which is chromatographed on silica 0.04-0.06 mm in CH2Cl2/MeOH: 90/10 then 80/20. In this way the expected product is isolated in the form of a yellow resin of 242 mg in weight.

Analyses:

IR spectrum:—Absorption OH/NH region —C═O: 1647 cm−1; —C═C+aromatic: 1597, 1583, 1570, 1508, 1498 cm−1. ¹H NMR spectrum: DMSO D⁶ δ (ppm) 1.51(bd), 3.09(bd): 2H; 2.98(bd), 2.15(bt): 2H; 2.98(bd), 2.39(masked): 2H; 3.90(s), 3.94(s): 6H; 2.32(s): CH3-N; 3.37(bd): 1H(ax); 3.82(s): 1H(eq); 6.66(s): 1H; 6.73(s): 1H; 7.56, 8.23 AA′BB′: 4H; 4.54: 1H(OH).

Stage b): 5,7-dihydroxy-8-[(3S,4R)-3-hydroxy-1-methyl-4-piperidinyl]-2-[4-(trifluoromethoxy)-phenyl]-4H-benzopyran-4-one trifluoroacetate

144 mg of the product obtained in Stage a) above (0.30 mmol), 3 cm3 of anhydrous DMF and 450 mg of Reillex-pyridine-HI (≈4 mmol/g) (1.8 mmol-6 eq.) obtained as indicated in Preparation 2) above are introduced, under N2, into a 20 cm3 flask with a reflux condenser. The reaction medium is taken to 120° C. for 19 hours and to 150° C. for 24 hours then returned to ambient temperature, filtered on Iéna and washed with 10 cm3 of CH3CN+3 cm3 of MeOH, then 1.2 g of PTBD resin (2.6 mmol/g) (3.1 mmol-10 eq.) is added to the filtrate. Agitation is carried out overnight at ambient temperature, followed by filtering on Iéna, washing with CH3CN then releasing with CH3CN/TFA: 95/05 under agitation, filtering on Iéna, rinsing with CH3CN/TFA: 95/05 then bringing to dryness under vacuum. 171 mg of a yellow varnish is recovered which is taken up in 6 cm3 of HPLC eluent described hereafter, filtered on a microporous filter-0.45 μm then injected 3 times into preparative HPLC: Kromasil C18—10 μm—500×22 mm λ=225 nm—Eluent: CH3CN/H2O/TFA: 35/65/0.1-15 ml/min.

The appropriate fractions are combined and concentrated under vacuum then lyophilized. In this way the expected product is isolated in the form of a pale yellow solid of 77 mg in weight.

Analyses:

¹H NMR spectrum: DMSO D⁶ δ (ppm) 1.82(bd), 3.18(m): 2H; 3.19(m), 3.49(masked): 2H; 3.42(bs): 2H; 3.55(masked): 1H(ax); 2.83(d): CH3-N; 4.09(bs): 1H(eq); 6.37(s): 1H; 7.00(s): 1H; 7.58, 8.27 AA′BB′: 4H; 9.43, 11.27, 13.11, 5.98(bs): 4H(mobile).

### EXAMPLE 25

### trifluoroacetate of 4-[5,7-dihydroxy-8-[(3S,4R)-3-hydroxy-1-methyl-4-piperidinyl]-4-oxo-4H-benzopyran-2-yl]-benzoic acid

Stage a): methyl 4-[5,7-dimethoxy-8-[(3S,4R)-3-hydroxy-1-methyl-4-piperidinyl]-4-oxo-4H-benzopyran-2-yl]-benzoate

1) Condensation: methyl 4-[3-[2-hydroxy-4,6-dimethoxy-3-[(3S,4R)-3-hydroxy-1-methyl-4-piperidinyl]phenyl]1,3-dioxopropyl]-benzoate

196 mg of NaH at 50% in oil (M=24) (8 mmol-4 eq.), 5 cm3 of DMSO/NK20 and 309 mg of Acetoflocinopiperidol (1 mmol) are introduced, under N2, into a 100 cm3 flask. The reaction medium is agitated for 1 hour at ambient temperature, then 582 mg of dimethyl 1,4-benzenedicarboxylate (M=194.1) (3 mmol-3 eq.) is introduced and the whole is maintained under agitation for 20 hours.

2) Cyclization: methyl 4-[5,7-dimethoxy-8-[(3S,4R)-3-hydroxy-1-methyl-4-piperidinyl]-4-oxo-4H-benzopyran-2-yl]-benzoate 2.5 cm3 of 36% HCl (0.03 mmol-30 eq.) is added to the reaction medium obtained in 1) above. The medium is taken to 50° C. for 2.5 hours, then left for 48 hours at ambient temperature. The reaction medium is treated by diluting with 5 cm3 of water. 2.5 cm3 of 33% NaOH is added dropwise, followed by extracting with 60 cm3 of CH2Cl2/MeOH: 9/1, washing with water, drying over anhydrous Na2SO4 and bringing to dryness under vacuum. 314 mg of a brown oil is obtained which is chromatographed on silica 0.04-0.06 mm in CH2Cl2/MeOH: 80/20. In this way the expected product is isolated in the form of a yellow solid of 220 mg in weight.

Analyses:

IR spectrum:—Absorption OH/NH region —C═O: 1722 cm−1; 1647 cm−1; conjugated system+aromatic: 1619, 1596, 1574, 1494 cm−1. ¹H NMR spectrum: CDCl3 δ (ppm) 1.63(m), 3.11: 2H; 2.19, 3.21: 2H; 2.39, 3.21: 2H; 2.44(s): N—CH3; 3.56(ddd): 1H(ax); 4.05(b): 1H(eq); 3.45: 1H (OH); 3.96(s), 3.97(s), 4.50(s): 9H; 6.44(s), 6.69(s): 2; 7.89, 8.18: 4H.

Stage b): trifluoroacetate of 4-[5,7-dihydroxy-8-[(3S,4R)-3-hydroxy-1-methyl-4-piperidinyl]-4-oxo-4H-benzopyran-2-yl]-benzoic acid

90 mg of the dimethoxy obtained in Stage a) above (0.198 mmol), 1.9 cm3 of anhydrous DMF and 1238 mg of Reillex -pyridine-HI (≈4 mmol/g) (4.87 mmol-25 eq.) obtained as indicated above in Preparation 2) are introduced, under N2, into a 30 cm3 flask with a reflux condenser. The reaction medium is taken to 130° C. for approximately 60 hours then to 140° C. for 5 hours, then returned to ambient temperature, diluted with MeOH+DMF, filtered on Iéna, rinsed with DMF, the filtrate is brought to dryness under vacuum with a rotary evaporator. The oily residue is taken up in 4 cm3 of the HPLC eluent: MeOH/H2O/TFA: 55/45/0.1—15 ml/min, filtered on a microporous filter-0.45 μm then injected 4 times into preparative HPLC: Kromasil C18—10 μm—500×22 mm—λ=225 nm. The appropriate fractions are combined and concentrated under vacuum then lyophilized. In this way the expected product is isolated in the form of a yellow solid of 20 mg in weight.

Analyses:

¹H NMR spectrum: DMSO D⁶ δ (ppm) 3.41: 2H; 4.09: 1H; 3.54: 1H; 1.80, 3.18: 2H; 3.18, 3.46: 2H; 2.83: N—CH3; 7.04: 1H; 6.37: 1H; 8.14, 8.24: 4H; 5.97, 9.34, 11.26, 13.08: 4H (mobile).

### EXAMPLE 26

### 2-[3-[(4-bromo-3,5-dimethyl-1H-pyrazol-1-yl) methyl]-phenyl]-5,7-dihydroxy-8-[(3S,4R)-3-hydroxy-1-methyl-4-piperidinyl]-4H-benzopyran-4-one trifluoroacetate

Stage a): 2-[3-[(4-bromo-3,5-dimethyl-1H-pyrazol-1-yl)methyl]-phenyl]-5,7-dimethoxy-8-[(3S,4R)-3-hydroxy-1-methyl-4-piperidinyl]-4H-benzopyran-4-one

1) Condensation: 1-[3-[(4-bromo-3,5-dimethyl-1H-pyrazol-1-yl)methyl]-phenyl]-3-[2-hydroxy-4,6-dimethoxy-3-[(3S,4R)-3-hydroxy-1-methyl-4-piperidinyl]phenyl]-1,3-propanedione 250 mg of NaH at 50% in oil (M=24) (5.2 mmol-2.7 eq.), 6.5 cm3 of DMSO/NK20 and 600 mg of Acetoflocinopiperidol (1.9 mmol) are introduced, under N2, into a flask. Agitation is carried out for 1 hour at ambient temperature, then 1.460 g of methyl 3-[(4-bromo-3,5-dimethyl-1H-pyrazol-1-yl)methyl]-benzoate (M=323.19) (4.5 mmol-2.3 eq.) is introduced and the reaction medium is maintained under agitation for 22 hours at ambient temperature, then heated at 75° C. before returning to ambient temperature for 18 hours.

2) Cyclization: 2-[3-[(4-bromo-3,5-dimethyl-1H-pyrazol-1-yl)methyl]-phenyl]-5,7-dimethoxy-8-[(3S,4R)-3-hydroxy-1-methyl-4-piperidinyl]-4H-benzopyran-4-one

2 cm3 of 36% HCl (24 mmol-12 eq.) is added to the reaction medium obtained above in 1). The reaction medium is then taken to 50° C. for 1 hour 40 minutes then another 2 cm3 of 36% HCl (24 mmol-12 eq.) is added. The reaction medium is maintained for another 2 hours at 50° C. then returned to ambient temperature, poured into 60 cm3 of NaOH(N), extracted with 2×100 cm3 of CH2Cl2/MeOH: 95/5, washed with a saturated solution of NaCl, dried over anhydrous Na2SO4 and brought to dryness under vacuum. 1.23 g of a yellow resin is obtained which is chromatographed on silica 0.04-0.06 mm in CH2Cl2/MeOH: 90/10. In this way the expected product is isolated in the form of yellow foam of 526 mg in weight.

Analyses:

¹H NMR spectrum: CDCl3 δ (ppm) 1.66(d), 3.22(masked): 2H; 2.33(bt), 3.19(masked): 2H; 2.52(masked), 3.28(masked): 2H; 3.57: 1H(ax); 4.09(bs): 1H(eq); 2.21(s), 2.24(s): 6H; 2.52(s): N—CH3; 3.98(s), 4.00(s): 6H; 6.45(s): 1H; 6.55(s): 1H; 5.29(s): 2H; 7.16(d): 1H; 7.48(t): 1H; 7.61(bs): 1H; 7.78(d): 1H.

Stage b): 2-[3-[(4-bromo-3,5-dimethyl-1H-pyrazol-1-yl)methyl]-phenyl]-5,7-dihydroxy-8-[(3S,4R)-3-hydroxy-1-methyl-4-piperidinyl]-4H-benzopyran-4-one trifluoroacetate 150 mg of the dimethoxy (0.26 mmol) obtained in Stage a) above, 2.6 cm3 of anhydrous DMF and 710 mg of Reillex-pyridine-HI (≈4 mmol/g) (2.8 mmol-11 eq.) obtained as indicated above in Preparation 2) are introduced, under N2, into a 20 cm3 flask with a reflux condenser. The reaction medium is taken to 130° C. for 30 hours, then returned to ambient temperature, diluted with 10 cm3 MeOH, the pH is adjusted to 8 with a saturated solution of NaOH in MeOH, the Reillex filtered on a 20 μm filtration cartridge+microporous filter 0.5 μm and brought to dryness under high vacuum. 532 mg of a cream solid is recovered which is taken up in the HPLC eluent: MeOH/H2O/TFA: 65/35/0.1, filtered on a microporous filter-0.45 μm then injected into preparative HPLC: Kromasil C18—10 μm—500×22 mm—λ=225 nm—15 ml/min. The appropriate fractions are combined and concentrated under vacuum then lyophilized. In this way the expected product is isolated in the form of a yellow solid of 38 mg in weight.

Analyses:

¹H NMR spectrum: DMSO D⁶ δ (ppm) 2.81(s): N—CH3; 2.11(s), 2.24(s): 6H; 1.86, 3.18: 2H; 3.49, 3.20: 2H; 3.38: 2H; 4.11(bs): 1H(ax); 3.52: 1H (eq); 5.39: 2H; 6.37(s): 1H; 6.89(s): 1H; 7.31(d): 1H; 7.59(t): 1H; 7.93(bs): 1H; 8.03(d): 1H; 13.11, 11.2, 9.44, 6.0: 4H (mobile).

### EXAMPLE 27

### 5,7-dihydroxy-2-[3-[(3,5-dimethyl-1H-pyrazol-1-yl)methyl]-phenyl]-8-[(3S,4R)-3-hydroxy-1-methyl-4-piperidinyl]-4H-benzopyran-4-one trifluoroacetate

The operation is carried out as in Example 26 and at the same time the product of Example 27 is obtained. In this way the expected product is isolated in the form of a yellow solid of 74 mg in weight.

Analyses:

¹H NMR spectrum: DMSO D⁶ δ (ppm) 2.81(s): N—CH3; 2.10(s), 2.21(s): 6H; 3.19(bt), 1.86(bd): 2H; 3.51(bt), 3.20: 2H; 3.40: 2H; 4.10 (masked): 1H(ax); 3.53(bt): 1H(eq); 5.32: 2H; 6.36(s): 1H; 6.86(s): 1H; 7.27(d): 1H; 7.57(t): 1H; 7.88(s): 1H; 8.01(d): 1H; 13.11, 11.20, 9.42, 5.96: 4H (mobile).

### EXAMPLE 28

### 2-[2,5-bis(2,2,2-trifluoroethoxy)-phenyl]-5,7-dihydroxy-8-[(3S,4R)-3-hydroxy-1-methyl-4-piperidinyl]-4H-benzopyran-4-one trifluoroacetate

Stage a): 2-[2,5-bis(2,2,2-trifluoroethoxy)-phenyl]-5,7-dimethoxy-8-[(3S,4R)-3-hydroxy-1-methyl-4-piperidinyl]-4H-benzopyran-4-one

1) Condensation: 1-[2,5-bis(2,2,2-trifluoroethoxy)-phenyl]-3-[2-hydroxy-4,6-dimethoxy-3-[(3S,4R)-3-hydroxy-1-methyl-4-piperidinyl]phenyl]-1,3-propanedione

196 mg of NaH at 50% in oil (M=24) (4 mmol-4 eq.), 5 cm3 of DMSO/NK20 and 309 mg of Acetoflocinopiperidol (1 mmol) are introduced, under N2, into a 30 cm3 flask. Agitation is carried out for 1 hour at ambient temperature, then 996 mg of methyl 2,5-bis(2,2,2-trifluoroethoxy)-benzoate (M=332.2) (3 mmol 3 eq.) is introduced. The reaction medium is maintained under agitation for 20 hours.

2) Cyclization: 2-[2,5-bis(2,2,2-trifluoroethoxy)-phenyl]-5,7-dimethoxy-8-[(3S,4R)-3-hydroxy-1-methyl-4-piperidinyl]-4H-benzopyran-4-one

2.5 cm3 of 36% HCl (24 mmol-24 eq.) is added to the reaction medium obtained above in 1). The reaction medium is then taken to 50° C. for 3 hours 30 minutes, returned to ambient temperature, 3 cm3 of conc. NaOH is added, followed by extracting 3 times with 20 cm3 of CH2Cl2/MeOH: 9/1, washing with water, drying over anhydrous MgSO4 and bringing to dryness under vacuum. 1 g of a brown oil is obtained which is chromatographed on silica 0.04-0.06 mm in CH2Cl2/MeOH: 80/20. In this way the expected product is isolated in the form of a yellow solid of 146 mg in weight.

Analyses:

¹H NMR spectrum: CDCl3 δ (ppm) 1.61(m), 3.26(m): 2H; 2.23(m), 3.14(m): 2H; 2.42(bs): N—CH3; 2.44(masked), 3.19(m): 2H; 3.41(b): 1H (OH); 3.51(ddd): 1H(ax); 4.03(bs): 1H(eq); 3.97(s): 6H; 4.39(qd), 4.44(qd), 4.67(m): 4H; 6.43(s): 1H; 6.75(s): 1H; 6.99(d): 1H; 7.07(dd): 1H; 7.64 (bd) : 1H.

Stage b): 2-[2,5-bis(2,2,2-trifluoroethoxy)-phenyl]-5,7-dihydroxy-8-[(3S,4R)-3-hydroxy-1-methyl-4-piperidinyl]-4H-benzopyran-4-one trifluoroacetate

130 mg of the dimethoxy obtained above in a) (0.25 mmol), 15 cm3 of anhydrous DMF and 682 mg of Reillex-pyridine -HI Pyridine (≈4 mmol/g) (2.72,mmol-11 eq.) obtained as indicated above in Preparation 2) are introduced, under N2, into a 30 cm3 flask with a reflux condenser. The reaction medium is taken to 150° C. for 36 hours, then returned to ambient temperature, diluted with CH3CN, filtered on Iéna, rinsed with CH3CN then brought to dryness under vacuum. A crude product is obtained of 650 mg in weight. The crude product obtained is solubilized in 10 cm3 of CH3CN, 10 cm3 of MeOH and 1.9 g of PTBD resin (2.6 mmol/g) (5 mmol-20 eq.). Agitation is carried out for 20 hours at ambient temperature, followed by filtering on Iéna, washing with CH3CN then releasing with 12 cm3 of CH3CN/TFA: 95/05 under agitation, filtering on Iéna, rinsing with CH3CN/TFA: 95/05 then bringing to dryness under vacuum. 150 mg of an amorphous orange product is recovered which is taken up in 7.5 cm3 of HPLC eluent CH3CN/H2O/TFA: 42/58/0.1, filtered on a microporous filter-0.45 μm then injected 5 times into preparative HPLC: Kromasil C18—10 μm—500×22 mm—λ=225 nm—15 ml/min. The appropriate fractions are combined and concentrated under vacuum then lyophilized. In this way the expected product is isolated in the form of a yellow solid of 45 mg in weight.

Analyses:

¹H NMR spectrum: DMSO D⁶ δ (ppm) 1.91(bd), 3.07(m): 2H; 3.12(masked), 3.36(m): 2H; 3.24(m), 3.32(masked): 2H; 2.74(bs): N—CH3; 4.12(bs), 4.04(bs): 1H(eq); 3.47(masked): 1H(ax); 4.89(q): 4H; 6.10(bs), 5.83(bs): 1H (OH); 6.36(s): 1H; 6.65(s): 1H; 7.37(masked), 7.46(d): 3H; 9.32, 11.22(bs), 13.05(b), 13.14(s): 4H (mobile).

### EXAMPLE 29

### 5,7-dihydroxy-8-[(3S,4R)-3-hydroxy-1-methyl-4-piperidinyl]-2-(3-isoquinolinyl)-4H-benzopyran-4-one

Stage a): 5,7-dimethoxy-8-[(3S,4R)-3-hydroxy-1-methyl-4-piperidinyl]-2-(3-isoquinolinyl)-4H-benzopyran-4-one

5 1) Condensation: 1-[2-hydroxy-4,6-dimethoxy-3-[(3S,4R)-3-hydroxy-1-methyl-4-piperidinyl]phenyl]-3-(3-isoquinolinyl) 1,3-propanedione

124 mg of NaH at 50% in oil (M=24) (2.6 mmol-4 eq.), 6.5 cm3 of DMSO/NK20 and 200 mg of Acetoflocinopiperidol (0.65mmol) by fractions are introduced, under Ar, into a 30 cm3 tube. Agitation is carried out for 1 hour at ambient temperature, then 363 mg of methyl 3-isoquinolinecarboxylate (M=187.2) (11.9 mmol-3 eq.) is introduced and the reaction medium is maintained under agitation for 18 hours.

2) Cyclization: 5,7-dimethoxy-8-[(3S,4R)-3-hydroxy-1-methyl-4-piperidinyl]-2-(3-isoquinolinyl)-4H-benzopyran-4-one 4.4 cm3 of 36% HCl (51 mmol-79 eq.) is added to the reaction medium obtained above in 1), and the medium is then taken to 50° C. for 3 hours 30 minutes, returned to ambient temperature, poured into 60 cm3 of NaOH(N), extracted with 2×50 cm3 of CH2Cl2/MeOH: 9/1, washed with 50 cm3 H2O, dried over anhydrous Na2SO4 and brought to dryness under vacuum. 533 mg of a pale yellow solid is obtained which is chromatographed on silica 0.04-0.06 mm in CH2Cl2/MeOH: 90/10. In this way the expected product is isolated in the form of a pale yellow solid of 294 mg in weight.

Analyses:

IR spectrum: absorption OH/NH region —C═O: 1642 cm−1; conjugated system+aromatic: 1623, 1615, 1596, 1575, 1563, 1492 cm−1.

¹H NMR spectrum: DMSO D⁶ δ (ppm) 1.90, 3.43: 2H; 2.83(bs): N—CH3; 3.42: 2H; 3.44: 2H; 3.71(bd): 1H(ax); 4.16(bs): 1H(eq); 3.92(s), 3.96(s): 6H; 5.47(b): 1H (OH); 6.70(s): 1H; 7.07(s): 1H; 7.84(ddd): 1H; 7.94(ddd): 1H; 8.26(d): 1H; 8.35(bd): 1H; 8.54(s): 1H; 9.47(s): 1H.

Stage b): 5,7-dihydroxy-8-[ (3S,4-R)-3-hydroxy-1-methyl-4-piperidinyl]-2-(3-isoquinolinyl)-4H-benzopyran-4-one 168 mg of the dimethoxy obtained above in a) (0.38 mmol), 4 cm3 of anhydrous DMF and 470 mg of Reillex-pyridine-HI (≈4 mmol/g) (1.9 mmol-5 eq.) obtained as indicated above in Preparation 2) are introduced, under Ar, into a flask with a reflux condenser. The reaction medium is taken to 150° C. for 16 hours 30 minutes, then returned to ambient temperature, filtered on a microporous filter-0.45 μm then adjusted to pH=8-9 by adding 0.5 cm3 of NaOH(N), diluted with 20 cm3 H2O, agitated for 1 hour at ambient temperature, filtered on Iéna, washed with 10 cm3 of H2O. The solid is redispersed in H2O then the medium is diluted with 15 cm3 of DMF. The reaction medium is taken to reflux then left to return to ambient temperature before placing the medium in a refrigerator for 4 days to crystallize. After returning to ambient temperature, filtering is carried out on Iéna, followed by washing with water and drying under vacuum at 60° C. In this way the expected product is isolated in the form of a yellow solid of 56 mg in weight.

Analyses:

¹H NMR spectrum: DMSO D⁶ δ (ppm) 1.98, 3.32: 2H; 2.89(d): N—CH3; 3.47: 2H; 3.38, 3.58: 2H; 3.67(bd): 1H(ax); 4.22(bs): 1H(eq); 6.38(s): 1H; 7.26(s): 1H; 7.88(bt): 1H; 7.98(bt): 1H; 8.28(bd): 1H; 8.37(bd): 1H; 8.60(s): 1H; 9.50(s): 1H; 11.23, 13.11: 3H; 9.48(d): 1H.

### EXAMPLE 30

### 2-(2-chlorophenyl)-5,7-dihydroxy-6-[(3S,4R)-3-hydroxy-1-methyl-4-piperidinyl]-4H-benzopyran-4-one trifluoroacetate

466 mg of 2-(2-chlorophenyl)-5,7-dimethoxy-8-[(3S,4R)-3-hydroxy-1-methyl-4-piperidinyl]-4H-benzopyran-4-one hydrochloride prepared as indicated in the Patent Application number FR 9807677 (10 mmol), 1.1 cm3 of 48% HBr (d=1.49) (10 mmol-1 eq.), 2.4 cm3 of H2O are introduced, under N2, into a 10 cm3 flask with a reflux condenser and this suspension is taken to 140° C. for 21 hours and to 160° C. for 2 hours 30 minutes.

Then another 1.2 cm3 of 48% HBr (10 eq.) is added and the reaction medium is heated to 160° C. for another 31 hours, then returned to ambient temperature, diluted with H2O, the precipitate is redissolved in CH3CN, then 7.7 g of PTBD resin (2.6 mmol/g) (20 mmol-20 eq.) is added. Agitation is carried out for 2 hours 30 minutes at ambient temperature, then another 2 g of PTBD resin (2.6 mmol/g) (5.2 mmol-5 eq.) is added. Agitation is carried out for another 1 hour 30 minutes at ambient temperature, followed by filtering on Iéna, washing with CH3CN then releasing with 40 cm3 of CH3CN/TFA: 95/05 under agitation, filtering on Iéna, rinsing with CH3CN/TFA: 95/05 then bringing to dryness under vacuum. 535 mg of a yellow resin is recovered which is taken up in 10 cm3 of HPLC eluent CH3CN/H2O/TFA: 35/65/0.1, filtered on a microporous filter-0.45 μm then injected 10 times into preparative HPLC Kromasil C18—10 μm—500×22 mm—λ=225 nm—20 ml/min. The appropriate fractions are combined and concentrated under vacuum then lyophilized. In this way the expected product is isolated in the form of a yellow solid of 68.3 mg in weight.

Analyses:

¹H NMR spectrum: DMSO D⁶ δ (ppm) 2.76(s): N—CH3; 1.73, 3.12: 2H; 3.10, 3.40: 2H; 3.32: 2H; 3.40: 1H; 4.11(bs): 1H(eq); 6.51(s): 1H; 6.59(s): 1H; 7.52(td), 7.61(td): 2H; 7.67(dd), 7.77(dd): 2H; 13.46: 1H (OH); 11.37: 1H (OH); 6.10, 9.40: 2H (mobile).

### EXAMPLE 31

### 2-(2-chlorophenyl)-5,7-dihydroxy-8-[(3S,4R)-3-hydroxy-1-methyl-4-piperidinyl]-4H-benzopyran-4-one trifluoroacetate

Example of the deprotection of hydroxyl functions using Reillex-HI-Pyridine in DMF

100 mg of 2-(2-chlorophenyl)-5,7-dimethoxy-8-[(3S,4R)-3-hydroxy-1-methyl-4-piperidinyl]-4H-benzopyran-4-one hydrochloride prepared as indicated in the Patent Application number FR 9807677 (0.21 mmol), 2 cm3 of anhydrous DMF and 581 mg of Reillex-pyridine-HI Pyridine (≈4 mmol/g) (2.2 mmol-11 eq.) obtained as indicated above in Preparation 2) are introduced, under N2, into a 30 cm3 flask with a reflux condenser. The reaction medium is taken to 130° C. for 48 hours and to 150° C. for 1 hour, then returned to ambient temperature, diluted with CH3CN, filtered on Iéna, rinsed with CH3CN then 1.5 g of PTBD resin (2.6 mmol/g) (4.2 mmol-20 eq.) is added. Agitation is carried out for 20 hours at ambient temperature, followed by filtering on Iéna, washing with CH3CN then releasing with 10 cm3 of CH3CN/TFA: 95/05 under agitation for 1 hour, filtering on Iéna, rinsing with CH3CN/TFA: 95/05 then bringing to dryness under vacuum. 130 mg of an amorphous orange product is recovered which is taken up in 3 cm3 of HPLC eluent: CH3CN/H2O/TFA: 35/65/0.1, filtered on a microporous filter-0.45 μm then injected 4 times into preparative HPLC: Kromasil C18—10 μm—500×22 mm—λ=225 nm—15 ml/min. The appropriate fractions are combined and concentrated under vacuum then lyophilized. In this way the expected product is isolated in the form of a yellow solid of 50 mg in weight.

Analyses:

MS/ESP: MH⁺=402⁺

### EXAMPLE 32

### 8-[(3S,4R)-3-acetyloxy-1-methyl-4-piperidinyl]-2-(chlorophenyl)5,7-dihydroxy-4H-benzopyran-4-one trifluoroacetate

Example of the deprotection of hydroxyl functions using HI-Pyridine in DMA.

115 mg (0.24 mmol) of 2-(2-chlorophenyl)-5,7-dimethoxy-8-[(3S,4R)-3-hydroxy-1-methyl-4-piperidinyl]-4H-benzopyran-4-one hydrochloride prepared as indicated in the Patent Application number FR 9807677, 2.6 cm3 of anhydrous DMA and 553 mg of HI-Pyridine−207.1) (2.64 mmol-11 eq.) obtained as indicated in Preparation 1) above are introduced, under N2, into a 30 cm3 flask with a reflux condenser. The reaction medium is taken to 130° C. for 20 hours, then returned to ambient temperature, diluted with CH3CN, then 1.9 g of PTBD resin (2.6 mmol/g) (4.9 mmol-20 eq.) is added. Agitation is carried out for 20 hours at ambient temperature, followed by filtering on Iéna, washing with CH3CN then releasing with 10 cm3 of CH3CN/TFA: 95/05 under agitation, filtering on Iéna, rinsing with CH3CN/TFA: 95/05 then bringing to dryness under vacuum. 330 mg of an amorphous orange product is recovered which is taken up in 6 cm3 of HPLC eluent: CH3CN/H2O/TFA: 35/65/0.1, filtered on a microporous filter-0.45 μm then injected 6 times into preparative HPLC: Kromasil C18—10 μm—500×22 mm—λ=225 nm—15 ml/min. The appropriate fractions are combined and concentrated under vacuum then lyophilized. In this way the expected product is isolated in the form of a yellow solid of 90 mg in weight.

Analyses:

MS/ESP: MH⁺=444⁺

### EXAMPLE 33

### Pharmaceutical Composition

Tablets were prepared corresponding to the following formula:

| | |
|---|---|
| Product of Example 2 | 0.2 g |
| Excipient for a tablet completed at | 1 g |
| (detail of excipient: lactose, talc, starch, | |
| magnesium stearate). | |

1) Products of formula (I): 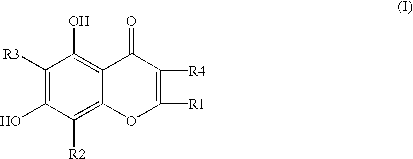
in which:
R1 represents a carbocyclic or heterocyclic monocyclic or bicyclic radical containing at most 12 members, saturated or unsaturated, containing one or more identical or different heteroatoms chosen from O, N, NH or S and can contain a —C(O) member,
the carbocyclic and heterocyclic radicals as defined above for R1, being optionally substituted by one or more radicals chosen from halogen atoms; the hydroxyl; cycloalkyl containing at most 6 members; acyl containing at most 7 carbon atoms; cyano; nitro; free, salified or esterified carboxy; tetrazolyl; —NH2, —NH(alk), —N(alk)(alk); SO2—NH—CO—NHR5 in which R5 represents an alkyl or phenyl radical; —C(O)—NH2, —C(O)—NH(alk), —C(O)—N(alk)(alk), —NH—C(O)—(alk), —N(alk)—C(O)—(alk); thienyl; phenyl; alkylphenyl; alkyl, alkenyl, alkylthio, alkoxy or phenoxy radicals themselves optionally substituted by one or more radicals chosen from halogen atoms, the —NH2, —NH(alk), —N(alk)(alk) radicals and the heterocyclic monocyclic or bicyclic radicals themselves optionally substituted by one or more radicals chosen from alkyl radicals and halogen atoms,
R2 and R3 are such that one represents a hydrogen atom and the other represents a piperidinyl radical optionally substituted by one or more hydroxyl and alkyl radicals,
R2 and R3 being alternatively able to take the same values in order to produce the corresponding isomers,
R4 represents a hydrogen atom, an alkyl or phenyl radical optionally substituted by one or more halogen atoms it being understood that in the above radicals, the alkyl and alkoxy radicals are linear or branched and contain at most 6 carbon atoms,
said products of formula (I) being in all possible racemic, enantiomeric and diastereoisomeric isomer forms, as well as the addition salts with mineral and organic acids or with mineral and organic bases of said products of formula (I).

2) Products of formula (I) as defined in claim 1 in which R2, R3 and R4 have the meanings indicated in claim 1 and R1 represents a phenyl, cyclohexyl or heterocyclic monocyclic or bicyclic radical containing 5 to 10 members, saturated or unsaturated, containing one or more identical or different heteroatoms chosen from O, N, NH or S and can contain a —C(O) member,
the phenyl, cyclohexyl and heterocyclic radicals as defined above for R1, being optionally substituted by one or more radicals chosen from halogen atoms; the hydroxyl; cyclohexyl, cyano; nitro; free, salified or esterified carboxy; tetrazolyl; —NH2, —NH(alk), —N(alk)(alk); SO2—NH—CO—NHR5 radicals in which R5 represents an alkyl or phenyl radical; phenyl; CF3; OCF3; alkyl, alkoxy and phenoxy radicals themselves optionally substituted by the pyrazolinyl radical itself optionally substituted by one or more radicals chosen from the alkyl radicals and halogen atoms,
it being understood that in the above radicals, the alkyl and alkoxy radicals are linear or branched and contain at most 6 carbon atoms,
said products of formula (I) being in all possible racemic, enantiomeric and diastereoisomeric isomer forms, as well as the addition salts with mineral and organic acids or with mineral and organic bases of said products of formula (I).

3) Products of formula (I) as defined in claim 1 or 2 in which R2, R3 and R4 have the meanings indicated in claim 1 and R1 represents a phenyl, cyclohexyl, pyrazolinyl, pyridyl, furyl, thienyl, isoxazolyl, isoquinolyl or quinolyl radical, these radicals being optionally substituted by one or more radicals chosen from halogen atoms; the cyclohexyl; cyano; nitro; hydroxyl; free, salified or esterified carboxy; tetrazolyl; —NH2, —NH(alkyl), —N(alkyl)(alkyl); SO2—NH—CO—NHR5 radical in which R5 represents an alkyl or phenyl radical; phenyl; alkyl, alkoxy or phenoxy; CF3; OCF3; pyrazolinyl radical itself optionally substituted by one or more radicals chosen from the alkyl radicals and halogen atoms,
it being understood that in the above radicals, the alkyl and alkoxy radicals are linear or branched and contain at most 4 carbon atoms,
said products of formula (I) being in all possible racemic, enantiomeric and diastereoisomeric isomer forms, as well as the addition salts with mineral and organic acids or with mineral and organic bases of said products of formula (I).

4) Products of formula (I) as defined in any one of claims 1 to 3 in which R1 has the meaning indicated in any one of claims 1 to 3, R2 and R3 are such that one represents the hydrogen atom and the other represents a piperidinyl radical optionally substituted by a hydroxyl radical on a carbon-containing member and an alkyl radical on the nitrogen atom and R4 represents a hydrogen atom,
said products of formula (I) being in all possible racemic, enantiomeric and diastereoisomeric isomer forms, as well as the addition salts with mineral and organic acids or with mineral and organic bases of said products of formula (I).

5) The following products of formula (I):
2-(2-chloro-4-fluorophenyl)-5,7-dihydroxy-8-[(3S,4R)-3-hydroxy-1-methyl-4-piperidinyl]-4H-benzopyran-4-one trifluoroacetate
2-(2,5-dichloro-3-thienyl)-5,7-dihydroxy-8-[(3S,4R)-3-hydroxy-1-methyl-4-piperidinyl]-4H-benzopyran-4-one trifluoroacetate
5,7-dihydroxy-8-[(3S,4R)-3-hydroxy-1-methyl-4-piperidinyl]-2-(5-methyl-3-isoxazolyl)-4H-benzopyran-4-one trifluoroacetate
5,7-dihydroxy-8-[(3S,4R)-3-hydroxy-1-methyl-4-piperidinyl]-2-[5-(trifluoromethyl)-1-phenyl-1H-pyrazol-4-yl]-4H-benzopyran-4-one trifluoroacetate
5,7-dihydroxy-6-[(3S,4R)-3-hydroxy-1-methyl-4-piperidinyl]-2-[3-(phenoxy)-phenyl]-4H-benzopyran-4-one trifluoroacetate

6) Process for the preparation of the products of formula (I), as defined in claim 1, characterized in that the compound of formula (II): 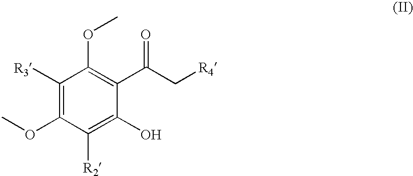
in which R2′, R3′ and R4′ have the meanings indicated in claim 1 for R2, R3 and R4 respectively, in which the optional reactive functions are optionally protected by protective groups
is subjected to a reaction with a compound of formula (III):
R1′COX (III)
in which R1′ has the meaning indicated in claim 1 for R1, in which the optional reactive functions are optionally protected by protective groups and X represents a halogen atom or an alkoxy radical containing at most 6 carbon atoms,
in order to obtain the product of formula (IV): 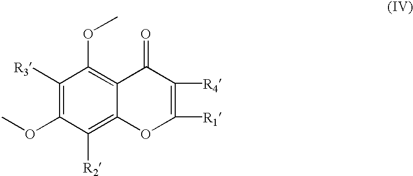
in which R1′, R2′, R3′ and R4′ have the meanings indicated above,
which products of formula (IV) are subjected to a deprotection reaction of the hydroxyl radicals in order to obtain a product of formula (I′): 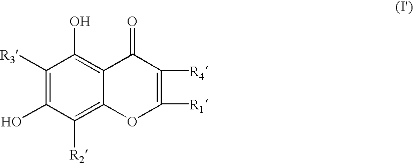
in which R1′, R2′, R3′ and R4′ have the meanings indicated above,
which products of formula (I′) can be products of formula (I) and which, in order to obtain some or other products of formula (I), can be subjected, if desired and if necessary, to one or more of the following conversion reactions, in any order:
a) an esterification reaction of an acid function,
b) a saponification reaction of the ester function to an acid function,
c) an oxidation reaction of the alkylthio group to a corresponding sulphoxide or sulphone,
d) a conversion reaction of the ketone function to an oxime function,
e) a reduction reaction of the free or esterified carboxy function to an alcohol function,
f) a conversion reaction of the alkoxy function to a hydroxyl function, or also of the hydroxyl function to an alkoxy function,
g) an oxidation reaction of the alcohol function to an aldehyde, acid or ketone function,
h) a conversion reaction of the nitrile radical to a tetrazolyl,
i) an elimination reaction of the protective groups which can be carred by the protected reactive functions,
j) a salification reaction by a mineral or organic acid or by a base in order to obtain the corresponding salt,
k) a resolution reaction of the racemic forms to resolved products,
said products of formula (I) thus obtained being in all possible racemic, enantiomeric and diastereoisomeric isomer forms.

7) Process for deprotection of the hydroxyl functions of a product of formula (IV) as defined claim 6: 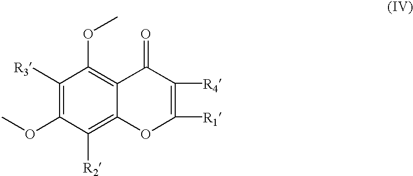
in which R1′, R2′, R3′ and R4′ have the meanings indicated claim 6,
characterized in that the product of formula (IV) is subjected either to the action of hydroiodic acid supported by a resin (such as for example Reillex-pyridine-TM-402 polymer) in an anhydrous polar solvent,
or to the action of a salt of hydroiodic acid such as Py-HI in an anhydrous polar solvent,
or to the action of concentrated hydroiodic acid, in order to obtain a product of formula (I′): 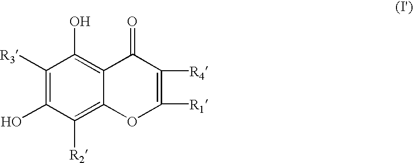
in which R1′, R2′, R3′ and R4′ have the meanings indicated in claim 6,
which products of formula (I′) can be the products of formula (I) and which, in order to obtain products or other products of formula (I), which, if desired and if necessary, can be subjected to one or more of the following conversion reactions, in any order:
a) an esterification reaction of the acid function,
b) a saponification reaction of the ester function to an acid function,
c) an oxidation reaction of the alkylthio group to a corresponding sulphoxide or sulphone,
d) a conversion reaction of the ketone function to an oxime function,
e) a reduction reaction of the free or esterified carboxy function to an alcohol function,
f) a conversion reaction of the alkoxy function to a hydroxyl function, or also the hydroxyl function to an alkoxy function,
g) an oxidation reaction of the alcohol function to an aldehyde, acid or ketone function,
h) a conversion reaction of the nitrile radical to a tetrazolyl,
i) an elimination reaction of the protective groups which can be carried by the protected reactive functions,
j) a salification reaction by a mineral or organic acid or by a base in order to obtain the corresponding salt,
k) a resolution reaction of the racemic forms to resolved products, said products of formula (I) thus obtained being in all possible racemic, enantiomeric and diastereoisomeric isomer forms.

8) As medicaments, the products of formula (I) as defined in claims 1 to 5, as well as the addition salts with pharmaceutically acceptable mineral and organic acids or mineral and organic bases of said products of formula (I).

9) As medicaments, the products of formula (I) as defined claim 5, as well as the addition salts with pharmaceutically acceptable mineral and organic acids or mineral and organic bases of said products of formula (I).

10) The pharmaceutical compositions containing as active ingredient, at least one of the medicaments as defined claims 8 and 9.

11) Pharmaceutical compositions according to claim 10 characterised in that they are used as antimitotic medicaments, in particular for chemotherapy for cancers or also for the treatment of psoriasis, parasitosis such as those due to fungi or to protists or Alzheimer's disease.

12) Pharmaceutical compositions according to claim 10 characterised in that they are used as antineurodegenerative medicaments in particular neuronal anti-apoptosis.

13) Use of the products of formula (I) as defined in claims 1 to 6 for the preparation of medicaments intended for the prevention or the treatment of diseases linked to a deregulation of the secretion and/or of the activity of protein tyrosine kinases.

14) Use of the products of formula (I) as defined in claims 1 to 5, for the preparation of medicaments intended for the chemotherapy for cancers, for the treatment of psoriasis, parasitosis such as those due to fungi or to protists, for the treatment of of Alzheimer's disease or for the treatment of neurodegeneratives affections in particular neuronal apoptosis.

15) As new industrial products, the compounds of formula (IV).
